(19) United States
(12) Patent Application Publication
(10) Pub. No.: US 2023/0125234 A1
(43) Pub. Date: Apr. 27, 2023

(54) ANTI CD44-CTLA4 BISPECIFIC ANTIBODIES

(71) Applicant: UCB BIOPHARMA SRL, Brussels (BE)

(72) Inventors: David Alan COOK, Slough, Berkshire (GB); Helen Margaret FINNEY, Slough, Berkshire (GB); Stephen Edward RAPECKI, Slough, Berkshire (GB)

(21) Appl. No.: 17/929,032

(22) PCT Filed: Feb. 13, 2020
(86) PCT No.: PCT/EP2020/053788

§ 371 Date: Aug. 10, 2022

(51) Int. Cl. C07K 16/28; A61P 35/00

(52) U.S. Cl. CPC C07K 16/2884; C07K 16/2818; A61P 35/00; C07K 2317/76; C07K 2317/622; C07K 2317/565; C07K 2317/31; C07K 2317/33

(57) ABSTRACT

The present disclosure relates to a bispecific anti CD44/anti CTLA4 antibody. The antibody construct has defined CDRs and variable chains sequences. The bispecific antibodies has improved activity on CDS and CD4 T cells compared to known antibodies.

### FIELD OF THE INVENTION

The present invention belongs to the field of multispecific antibodies binding at least CTLA4 and CD44, and their uses in the treatment of cancer and/or infectious diseases.

### BACKGROUND OF THE INVENTION

T cells are key to a successful cell-mediated immune response necessary to eliminate cancer cells, bacteria and viruses. They recognise antigens displayed on the surface of tumour cells or antigens from bacteria and viruses replicating within the cells or from pathogens or pathogen products endocytosed from the extracellular fluid. T cells have two major roles. They can become cytotoxic T cells capable of destroying cells marked as foreign. Cytotoxic T cells have a unique surface protein called CD8, thus they are often referred to as CD8+ T cells. Alternatively, T cells can become helper T cells, which work to regulate and coordinate the immune system. Helper T cells have a unique surface protein called CD4 and are thus often called CD4+ T cells. Helper T cells have several important roles in the immune system: 1) responding to activation by specific antigens by rapidly proliferating; 2) signaling B cells to produce antibodies; and 3) activating macrophages.

Cancer eludes the immune system by exploiting mechanisms developed to avoid auto-immunity. However, the immune system is programmed to avoid immune over-activation which could harm healthy tissue. T-cells activation is at the core of these mechanisms. Antigen specific T cells normally able to fight disease can become functionally tolerant (exhausted) to infectious agents or tumour cells by over stimulation or exposure to suppressive molecules. Therefore, molecules that enhance the natural function of T cells or overcome suppression of T cells have great utility in the treatment or prevention of cancer and infectious disease.

In recent years, immunotherapy has become an established treatment option for an increasing number of cancer patients, exemplified by the increased use of therapeutic antibody-based immune checkpoint inhibitors (CPI's). This has arisen from an increased immunological understanding of how cancer cells perturb immune cell activation by hijacking pathways normally involved in maintaining tolerance and skewing the balance between co-stimulation and co-inhibition (Chen and Mellman., Immunity. 39:1-105 (2013)). Amongst the pathways that have emerged as key regulators in this regard, include CTLA-4 and the PD-1/PD-L1 checkpoint molecules serving to down-regulate T cell and myeloid cell activation in the tumour microenvironment. Ipilimumab (anti-CTLA-4) was the first CPI to be approved in 2011 as a treatment for melanoma, closely followed by FDA approval of anti-PD1 directed antibodies, pembrolizumab and nivolumab in 2014 (Hargadon et al., International Immunopharmacol. 62:29-39 (2018)). There are still significant challenges in understanding differences in efficacy across patient groups, ranging from complete responses, to treatment relapse and even failure to respond, (Haslam and Prasad. JAMA Network Open.5:2e192535 (2019)). Many human solid tumours are associated with multiple cellular components of the immune system, and regulatory T cells (Tregs) represent a common component of this environment. Tregs are key players in limiting autoimmunity and maintaining immune homeostasis via suppression of CD4+(helper) and CD8+(cytotoxic) cells reacting to autologous, exogenous or cancer-associated antigens. Tregs are elevated within the tumour microenvironment and studies have shown that at least for some cancers, the density of tumour-infiltrating (intra-tumoral) Tregs correlates with cancer progression (Shang et al., Scientific reports. 5:15179-(2015)), suggesting a suppressive effect on tumour-specific T cell responses. This key role of Tregs in immune suppression and their elevated numbers in human cancers has been suggested as a potential major barrier to successful immunotherapy with current CPIs. Some experimental studies have suggested that anti-CTLA4 blockade for example, causes selective depletion of intra-tumoral Tregs, but the evidence in human cancer is limited.

Furthermore, the promising anti-tumour efficacy of several monoclonal antibodies, many cancers are refractory to treatments with a single antibody. Combinations of two or more antibodies are currently being tested in patients to provide improved methods of treatment. To date, these therapies rely on rational design of known mechanisms of action and are largely based on combining antigen-specificities known to be independently effective in the treatment of cancer, either as combination therapies or in a bispecific antibody format. This state of the art approach is a limiting factor in the development of new therapies as it relies on known therapies.

Identifying bispecific molecules that stimulate T cell activation in the presence of Tregs, and reversal of immune suppression, may represent novel and alternative therapeutic approaches for immunotherapy in cancer beyond conventional currently used CPIs.

### SUMMARY OF THE INVENTION

The present invention addresses the above-identified need by providing in a first aspect an antibody which comprises a first antigen-binding portion binding CTLA4 and a second antigen-binding portion binding CD44.

In one embodiment of this first aspect, each of the antigen-binding portions of the antibody which comprises a first antigen-binding portion binding CTLA4 and a second antigen-binding portion binding CD44 is a monoclonal antigen-binding portion.

In another embodiment, each of the antigen-binding portions is independently selected from a Fab, a Fab′, a scFv or a VHH. In yet another aspect, the antigen-binding portions are the antigen-binding portions of an IgG.

In another embodiment, the antibody which comprises a first antigen-binding portion binding CTLA4 and a second antigen-binding portion binding CD44 is chimeric, human or humanised, and preferably the antibody is humanised.

In another embodiment, the antibody comprises a heavy chain constant region selected from an IgG1, an IgG2, IgG3 or an IgG4 isotype, or a variant thereof.

In another embodiment, the antibody further comprises at least an additional antigen-binding portion. The additional antigen-binding portion may be capable of increasing the half-life of the antibody. Preferably the additional antigen-binding portion binds albumin, more preferably human serum albumin.

In another embodiment, the first antigen-binding portion binding CTLA4 of the antibody according to the present invention comprises a first heavy chain variable region and a first light chain variable region and the second antigen-binding portion binding CD44 comprises a second heavy chain variable region and a second light chain variable region and wherein:
a. The first heavy chain variable region comprises a CDR-H1 comprising SEQ ID NO: 21, a CDR-H2 comprising SEQ ID NO: 22 and a CDR-H3 comprising SEQ ID NO:
b. The first light chain variable region comprises a CDR-L1 comprising SEQ ID NO: 24, a CDR-L2 comprising SEQ ID NO: 25 and a CDR-L3 comprising SEQ ID NO: 26; and
c. The second heavy chain variable region comprises a CDR-H1 comprising SEQ ID NO: 27, a CDR-H2 comprising SEQ ID NO: 28 and a CDR-H3 comprising SEQ ID NO: 29; and
d. The second light chain variable region comprises a CDR-L1 comprising SEQ ID NO: 30, a CDR-L2 comprising SEQ ID NO: 31 and a CDR-L3 comprising SEQ ID NO: 32;
or
e. The first heavy chain variable region comprises SEQ ID NO: 33 and the first light chain variable region comprises SEQ ID NO: 35; and the second heavy chain variable region comprises SEQ ID NO: 37 and second light chain variable region comprises SEQ ID NO: 39;
or
f. The first heavy chain variable region is encoded by a nucleotide sequence comprising SEQ ID NO: 34 and the first light chain variable region is encoded by a nucleotide sequence comprising SEQ ID NO: 36; and the second heavy chain variable region is encoded by a nucleotide sequence comprising SEQ ID NO: 38 and second light chain variable region is encoded by a nucleotide sequence comprising SEQ ID NO: 40.

In a second aspect of the present invention there is provided a pharmaceutical composition comprising the antibody according to the first aspect of the invention and all its embodiments and one or more pharmaceutically acceptable excipients.

In a third aspect, the invention provides for the antibody according to the first aspect of the invention and all its embodiments or the pharmaceutical composition according to the second aspect of the invention and all its embodiments for use in therapy.

In one embodiment of this third aspect, the use is for the treatment of cancer and/or an infectious disease. In another embodiment, the antibody or the composition according to the invention and all its embodiments are for use in the treatment of cancer concomitantly or sequentially to one or more additional cancer therapies.

In another embodiment, the antibody for use in the treatment of cancer and/or an infectious disease is an antibody that stimulates T cell activation in the presence of regulatory T cells; wherein activation preferably reverses, at least in part or in full, immune suppression.

In a fourth aspect of the present invention, there is provided for a method for treating a subject afflicted with cancer and/or an infectious disease, comprising administering to the subject a pharmaceutically effective amount of the antibody according to the first aspect of the invention and all its embodiments or the pharmaceutical composition according to the second aspect of the invention and all its embodiments.

In one embodiment of this fourth aspect, the antibody or the composition are administered concomitantly or sequentially to one or more additional cancer therapies.

In one other embodiment, the method for treating a subject afflicted with cancer and/or an infectious disease, comprises administering to the subject a pharmaceutically effective amount of the antibody that stimulates T cell activation in the presence of regulatory T cells; wherein activation preferably reverses, at least in part or in full, immune suppression.

In a fifth aspect, the invention provides for the use of an antibody according to the first aspect of the invention and all its embodiments or the pharmaceutical composition according to the second aspect of the invention and all its embodiments in the manufacture of a medicament for treating cancer.

In one embodiment of this fifth aspect, the antibody or the composition are administered concomitantly or sequentially to one or more additional cancer therapies.

### BRIEF DESCRIPTION OF THE DRAWINGS

FIG. 1. General representation of a Fab-X and Fab-Y comprising antigen-binding portions and of the resulting bispecific antibody

FIG. 2. Median fluorescent intensity (MFI) values for unstimulated and anti-CD3 or SEB stimulated control wells. Peripheral blood mononuclear cell (PBMC) cultures were treated with superantigen Staphylococcus aureus Enterotoxin B (SEB) at 1 μg/mL or anti-CD3 (clone UCHT1) at 250 ng/mL or unstimulated for 48 hours. The conditioned media were collected and diluted 20-fold before analysis of the level of IL-2 using an IntelliCyt® QBead PlexScreen.

FIG. 3. Log 2 fold change in the MFI values of IL-2 levels in the culture media of PBMC cultures in the presence of SEB (1 μg/mL) stimulation. PBMC cultures were treated with SEB at 1 μg/mL for 48 hours in the presence of either the CD44-CTLA4 bispecific construct or control constructs. The conditioned media were collected and diluted 20-fold before analysis of the level of IL-2 using an IntelliCyt® QBead PlexScreen. Log 2 fold changes were calculated for the MFI of IL-2 levels in the treated samples relative to the SEB stimulated controls. N=4 donors, 2 technical replicates ±standard error of the mean (SEM).

FIG. 4. Log 2 fold change in the MFI values of IL-2 levels in the culture media of PBMC cultures in the presence of anti-CD3 (250 ng/mL) stimulation. PBMC cultures were treated with anti-CD3 (clone UCHT1) at 250 ng/mL for 48 hours in the presence of either the CD44-CTLA4 bispecific construct or control constructs. The conditioned media were collected and diluted 20-fold before analysis of the level of IL-2 using an IntelliCyt® QBead PlexScreen. Log 2 fold changes were calculated for the MFI of IL-2 levels in the treated samples relative to the anti-CD3 stimulated controls. N=4 donors, 2 technical replicates ±SEM.

FIG. 5. Log 2 fold change in the MFI values of IL-2 levels in the culture media of PBMC cultures in the absence of any other stimulation. PBMC were cultured for 48 hours in the presence of either the CD44-CTLA4 bispecific construct or control constructs. The conditioned media were collected and diluted 20-fold before analysis of the level of IL-2 using an IntelliCyt® QBead PlexScreen. Log 2 fold changes were calculated for the MFI of IL-2 levels in the treated samples relative to the unstimulated controls. N=4 donors, 2 technical replicates ±SEM.

FIG. 6. Log 2 fold change in the MFI values of IL-2 levels in the conditioned media of PBMC cultures in the presence of SEB (1 μg/mL) stimulation. PBMC cultures were treated with SEB at 1 μg/mL for 48 hours in the presence of either the CD44-CTLA4 bispecific construct or control constructs. The conditioned medias were collected and diluted 20-fold before analysis of the level of IL-2 using an IntelliCyt® QBead PlexScreen. Log 2 fold changes were calculated for the MFI values of IL-2 levels in the treated samples relative to the SEB stimulated controls. N=4 donors, 2 technical replicates ±SEM.

FIG. 7. Log 2 fold change in the MFI values of IL-2 levels in the conditioned media of PBMC cultures in the presence of anti-CD3 (250 ng/mL) stimulation. PBMC cultures were treated with anti-CD3 (UCHT1) at 250 ng/mL for 48 hours in the presence of either the CD44-CTLA4 bispecific construct or control constructs. The conditioned media were collected and diluted 20-fold before analysis of the level of IL-2 using an IntelliCyt® QBead PlexScreen. Log 2 fold changes were calculated for the MFI of IL-2 levels in the treated samples relative to the anti-CD3 stimulated controls. N=4 donors, 2 technical replicates ±SEM.

FIG. 8. Log 2 fold change in the MFI values of IL-2 levels in the conditioned media of PBMC cultures in the absence of any other stimulation. PBMC were cultured for 48 hours in the presence of either the CD44-CTLA4 bispecific construct or control constructs. The conditioned media were collected and diluted 20-fold before analysis of the level of IL-2 using an IntelliCyt® QBead PlexScreen. Log 2 fold changes were calculated for the MFI of IL-2 levels in the treated samples relative to the un stimulated controls. N=4 donors, 2 technical replicates ±SEM.

FIG. 9. Log 2 fold change in the median fluorescence intensity (MFI) of CD71 on CD8⁺ and CD4⁺ T cells in the presence of SEB at 100 ng/mL. Human PBMC were incubated in triplicate wells for 6 days with SEB plus 100 nM of the indicated antibodies. CD71 levels on the T cells was then measured by flow cytometry by gating on CD8⁺ (top plot) and CD4⁺ (bottom plot) populations. Log 2 fold changes were calculated for the MFI of CD71 levels in the treated samples relative to the SEB stimulated controls. N=4 donors ±SEM.

FIG. 10. Log 2 fold change in the concentration of IL-2 detected in the conditioned media of PBMC cultures stimulated with SEB (1 μg/mL). PBMC cultures were treated with SEB at 1 μg/mL for 48 hours in the presence of either the CD44-CTLA4 bispecific constructs or control construct. All Fab-K_{D}-Fabs tested had the same anti-CD44 Fab-X, combined with the different variable regions to CTLA4 (unique numeric identifiers) and the negative control arm (5599). The conditioned media was collected and diluted 20-fold before analysis of the level of IL-2 using a bead ELISA. Log 2 fold changes were calculated for the concentration of IL-2 levels in the treated samples relative to the SEB stimulated controls. N=2 donors ±SEM.

FIG. 11. Log 2 fold change in the MFI of CD25 (top) and CD71 (bottom) on CD4⁺ T_{EFF} cells in the presence expanded T_{REG} cells at a ratio of 1 T_{REG} to 4 T_{EFF} cells. The co-cultures were incubated in triplicate for 5 days with Suppression Inspector Beads (Miltenyi Biotec) plus 100 nM of the indicated antibodies. CD25 and CD71 levels on the T_{EFF} cells were then measured by flow cytometry by gating on CD4⁺, CTV⁺ populations. Log 2 fold changes were calculated for the MFI of CD25 and CD71 levels in the treated samples relative to the assay ratio controls. N=4 donors ±SEM.

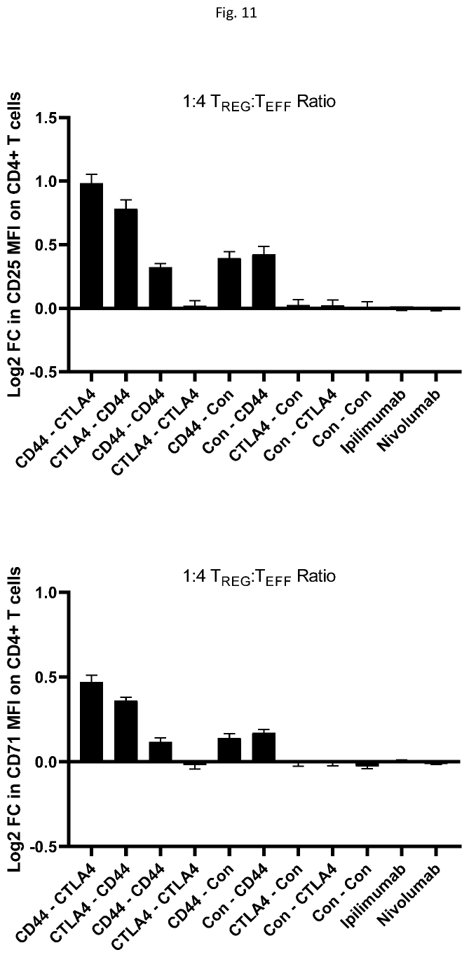

### DETAILED DESCRIPTION OF THE INVENTION

The present invention will now be described with respect to particular non-limiting aspects and embodiments thereof and with reference to certain figures and examples.

Technical terms are used by their common sense unless indicated otherwise. If a specific meaning is conveyed to certain terms, definitions of terms will be given in the context of which the terms are used.

Where the term “comprising” is used in the present description and claims, it does not exclude other elements. For the purposes of the present disclosure, the term “consisting of” is considered to be a preferred embodiment of the term “comprising of”.

Where an indefinite or definite article is used when referring to a singular noun, e.g. “a”, “an” or “the”, this includes a plural of that noun unless something else is specifically stated.

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject, i.e. a human, which may be predisposed to the disease but has not yet been diagnosed as having it; (b) inhibiting the disease, i.e., arresting its development; and (c) relieving the disease, i.e., causing regression of the disease.

A “therapeutically effective amount” refers to the amount of antibody comprising the distinct antigen-binding portions binding CTLA4 and CD44 that, when administered to a mammal or other subject for treating a disease, is sufficient to affect such treatment for the disease.

The present invention provides for antibodies comprising a first antigen-binding portion binding CTLA4 and a second antigen-binding portion binding CD44. The first and the second antigen-binding portions are located in the same antibody, i.e. they are part of the same polypeptide chain and/or associate via one or more covalent and/or non-covalent associations (such as the screening format Fab-Kd-Fab described herein or the classic heavy and light chain association forming a full IgG antibody) or are covalently linked so as to form one single molecule (such as cross-linking two separately expressed polypeptide chains, optionally via specific cross-linking agents).

CTLA4 and in particular human CTLA4 (Uniprot accession number P16410), also known as cytotoxic T lymphocyte protein 4 or CD152, is an inhibitory receptor acting as a major negative regulator of T cell responses. The affinity of CTLA4 for its natural B7 family ligands, CD80 and CD86, is considerably stronger than the affinity of their cognate stimulatory coreceptor CD28. CTLA4 is a key immune checkpoint like PD-1 which has been targeted by blocking antibodies in cancer patients to produce an effective anti-tumour response. The sequence of human CTLA4, including the signal peptide is shown as SEQ ID NO:1 (Table 1).

CD44 and in particular human CD44 (Uniprot accession number P16070), also known as LHR, MDU2, MDU3, MIC4, is a receptor for hyaluronic acid (HA) and mediates cell-cell and cell-matrix interactions through its affinity for HA, and possibly also through its affinity for other ligands such as osteopontin, collagens, and matrix metalloproteinases (MMPs). Through adhesion with HA, it plays an important role in cell migration, tumour growth and progression. In cancer cells, it may play an important role in invadopodia formation. It is also involved in lymphocyte activation, recirculation and homing, and in hematopoiesis. Altered expression or dysfunction causes numerous pathogenic phenotypes. The sequence of human CD44, including the signal peptide is shown as SEQ ID NO:2 (Table 1). Several isoforms are known due to alternative splicing and are identified herein as isoforms 1 to 19 (SEQ ID Nos: 2-20). Within the present application, antibodies were raised to isoform 4 having SEQ ID NO: 5. It differs from the canonical sequence (isoform 1 SEQ ID NO:2) at position 223 where a serine is replaced by a threonine in isoform 4 and by missing amino acids 224 to 266. Antibodies raised to this isoform will also recognise other isoforms of CD44. Within the present invention, unless specifically and explicitly mentioned, the term “CD44” is used to encompass all 19 isoforms of CD44, variants and splicing isoforms thereof generated by splicing out exons or alternative splicing within exons, and/or post-translational modification events

TABLE 1

| | |
|---|---|
| SEQ ID NO: 1 | MACLGFQRHKAQLNLATRTWPCTLLFFLLFIPVFCKAMHVAQPAVVLAS |
| Human CTLA4 | SRGIASFVCEYASPGKATEVRVTVLRQADSQVTEVCAATYMMGNELTF |
| | LDDSICTGTSSGNQVNLTIQGLRAMDTGLYICKVELMYPPPYYLGIGNG |
| | TQIYVIDPEPCPDSDFLLWILAAVSSGLFFYSFLLTAVSLSKMLKKRSPLT |
| | TGVYVKMPPTEPECEKQFQPYFIPIN |
| SEQ ID NO: 2 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 1 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATTLMSTSATATETATKRQETWDWF |
| | SWLFLPSESKNHLHTTTQMAGTSSNTISAGWEPNEENEDERDRHLSFS |
| | GSGIDDDEDFISSTISTTPRAFDHTKQNQDWTQWNPSHSNPEVLLQTT |
| | TRMTDVDRNGTTAYEGNWNPEAHPPLIHHEHHEEEETPHSTSTIQATP |
| | SSTTEETATQKEQWFGNRWHEGYRQTPKEDSHSTTGTAAASAHTSHP |
| | MQGRTTPSPEDSSWTDFFNPISHPMGRGHQAGRRMDMDSSHSITLQP |
| | TANPNTGLVEDLDRTGPLSMTTQQSNSQSFSTSHEGLEEDKDHPTTST |
| | LTSSNRNDVTGGRRDPNHSEGSTTLLEGYTSHYPHTKESRTFIPVTSAT |
| | GSFGVTAVTVGDSNSNVNRSLSGDQDTFHPSGGSHTTHGSESDGHS |
| | HGSQEGGANTTSGPIRTPQIPEWLIILASLLALALILAVCIAVNSRRRCGQ |
| | KKKLVINSGNGAVEDRKPSGLNGEASKSQEMVHLVNKESSETPDQFM |
| | TADETRNLQNVDMKIGV |
| SEQ ID NO: 3 | MDKFWWHAAWGLCLVPLSLAQIGVGRRKS |
| Isoform 2 | |
| SEQ ID NO: 4 | MDKFWWHAAWGLCLVPLSLAQIDLNITCRFAGVFHVEKNGRYSISRTE |
| human CD44 | AADLCKAFNSTLPTMAQMEKALSIGFETCRYGFIEGHWIPRIHPNSICA |
| Isoform 3 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGALMS |
| | TSATATETATKRQETWDWFSWLFLPSESKNHLHTTTQMAGTSSNTISA |
| | GWEPNEENEDERDRHLSFSGSGIDDDEDFISSTISTTPRAFDHTKQNQ |
| | DWTQWNPSHSNPEVLLQTTTRMTDVDRNGTTAYEGNWNPEAHPPLIH |
| | HEHHEEEETPHSTSTIQATPSSTTEETATQKEQWFGNRWHEGYRQTP |
| | KEDSHSTTGTAAASAHTSHPMQGRTTPSPEDSSWTDFFNPISHPMGR |
| | GHQAGRRMDMDSSHSITLQPTANPNTGLVEDLDRTGPLSMTTQQSNS |
| | QSFSTSHEGLEEDKDHPTTSTLTSSNRNDVTGGRRDPNHSEGSTTLLE |
| | GYTSHYPHTKESRTFIPVTSAKTGSFGVTAVTVGDSNSNVNRSLSGDQ |
| | DTFHPSGGSHTTHGSESDGHSHGSQEGGANTTSGPIRTPQIPEWLIILA |
| | SLLALALILAVCIAVNSRRRCGQKKKLVINSGNGAVEDRKPSGLNGEAS |
| | KSQEMVHLVNKESSETPDQFMTADETRNLQNVDMKIGV |
| SEQ ID NO: 5 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 4 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATSTSSNTISAGWEPNEENEDERDR |
| | HLSFSGSGIDDDEDFISSTISTTPRAFDHTKQNQDWTQWNPSHSNPEV |
| | LLQTTTRMTDVDRNGTTAYEGNWNPEAHPPLIHHEHHEEEETPHSTST |
| | IQATPSSTTEETATQKEQWFGNRWHEGYRQTPKEDSHSTTGTAAASA |
| | HTSHPMQGRTTPSPEDSSWTDFFNPISHPMGRGHQAGRRMDMDSSH |
| | SITLQPTANPNTGLVEDLDRTGPLSMTTQQSNSQSFSTSHEGLEEDKD |
| | HPTTSTLTSSNRNDVTGGRRDPNHSEGSTTLLEGYTSHYPHTKESRTFI |
| | PVTSAKTGSFGVTAVTVGDSNSNVNRSLSGDQDTFHPSGGSHTTHGS |
| | ESDGHSHGSQEGGANTTSGPIRTPQIPEWLIILASLLALALILAVCIAVNS |
| | RRRCGQKKKLVINSGNGAVEDRKPSGLNGEASKSQEMVHLVNKESSE |
| | TPDQFMTADETRNLQNVDMKIGV |
| SEQ ID NO: 6 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| Isoform 5 | TFSTVHPIPDEDSPWITDSTDRIPATTLMSTSATATETATKRQETWDWF |
| | SWLFLPSESKNHLHTTTQMAAGWEPNEENEDERDRHLSFSGSGIDDD |
| | EDFISSTISTTPRAFDHTKQNQDWTQWNPSHSNPEVLLQTTTRMTDVD |
| | RNGTTAYEGNWNPEAHPPLIHHEHHEEEETPHSTSTIQATPSSTTEETA |
| | TQKEQWFGNRWHEGYRQTPKEDSHSTTGTAAASAHTSHPMQGRTTP |
| | SPEDSSWTDFFNPISHPMGRGHQAGRRMDMDSSHSITLQPTANPNTG |
| | LVEDLDRTGPLSMTTQQSNSQSFSTSHEGLEEDKDHPTTSTLTSSNRN |
| | DVTGGRRDPNHSEGSTTLLEGYTSHYPHTKESRTFIPVTSAKTGSFGV |
| | TAVTVGDSNSNVNRSLSGDQDTFHPSGGSHTTHGSESDGHSHGSQE |
| | GGANTTSGPIRTPQIPEWLIILASLLALALILAVCIAVNSRRRCGQKKKLVI |
| | NSGNGAVEDRKPSGLNGEASKSQEMVHLVNKESSETPDQFMTADETR |
| | NLQNVDMKIGV |
| SEQ ID NO: 7 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 6 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATTLMSTSATATETATKRQETWDWF |
| | SWLFLPSESKNHLHTTTQMAGTSSNTISAGWEPNEENEDERDRHLSFS |
| | GSGIDDDEDFISSTISTTPRAFDHTKQNQDWTQWNPSHSNPEVLLQTT |
| | TRMTDVDRNGTTAYEGNWNPEAHPPLIHHEHHEEEETPHSTSTTASAH |
| | TSHPMQGRTTPSPEDSSWTDFFNPISHPMGRGHQAGRRMDMDSSHS |
| | ITLQPTANPNTGLVEDLDRTGPLSMTTQQSNSQSFSTSHEGLEEDKDH |
| | PTTSTLTSSNRNDVTGGRRDPNHSEGSTTLLEGYTSHYPHTKESRTFIP |
| | VTSAKTGSFGVTAVTVGDSNSNVNRSLSGDQDTFHPSGGSHTTHGSE |
| | SDGHSHGSQEGGANTTSGPIRTPQIPEWLIILASLLALALILAVCIAVNSR |
| | RRCGQKKKLVINSGNGAVEDRKPSGLNGEASKSQEMVHLVNKESSET |
| | PDQFMTADETRNLQNVDMKIGV |
| SEQ ID NO: 8 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 7 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATTLMSTSATATETATKRQETWDWF |
| | SWLFLPSESKNHLHTTTQMAGTSSNTISAGWEPNEENEDERDRHLSFS |
| | GSGIDDDEDFISSTISTTPRAFDHTKQNQDWTQWNPSHSNPEVLLQTT |
| | TRMTDVDRNGTTAYEGNWNPEAHPPLIHHEHHEEEETPHSTSTIQATP |
| | SSTTEETATQKEQWFGNRWHEGYRQTPKEDSHSTTGTAAASAHTSHP |
| | MQGRTTPSPEDSSWTDFFNPISHPMGRGHQAGRRMDMDSSHSITLQP |
| | TANPNTGLVEDLDRTGPLSMTTRNRNDVTGGRRDPNHSEGSTTLLEG |
| | YTSHYPHTKESRTFIPVTSAKTGSFGVTAVTVGDSNSNVNRSLSGDQD |
| | TFHPSGGSHTTHGSESDGHSHGSQEGGANTTSGPIRTPQIPEWLIILAS |
| | LLALALILAVCIAVNSRRRCGQKKKLVINSGNGAVEDRKPSGLNGEASK |
| | SQEMVHLVNKESSETPDQFMTADETRNLQNVDMKIGV |
| SEQ ID NO: 9 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 8 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATTLMSTSATATETATKRQETWDWF |
| | SWLFLPSESKNHLHTTTQMAGTSSNTISAGWEPNEENEDERDRHLSFS |
| | GSGIDDDEDFISSTISTTPRAFDHTKQNQDWTQWNPSHSNPEVLLQTT |
| | TRMTDVDRNGTTAYEGNWNPEAHPPLIHHEHHEEEETPHSTSTIQATP |
| | SSTTEETATQKEQWFGNRWHEGYRQTPKEDSHSTTGTAAASAHTSHP |
| | MQGRTTPSPEDSSWTDFFNPISHPMGRGHQAGRRMDMDSSHSITLQP |
| | TANPNTGLVEDLDRTGPLSMTTQQSNSQSFSTSHEGLEEDKDHPTTST |
| | LTSSRDQDTFHPSGGSHTTHGSESDGHSHGSQEGGANTTSGPIRTPQI |
| | PEWLIILASLLALALILAVCIAVNSRRRCGQKKKLVINSGNGAVEDRKPS |
| | GLNGEASKSQEMVHLVNKESSETPDQFMTADETRNLQNVDMKIGV |
| SEQ ID NO: 10 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 9 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATTLMSTSATATETATKRQETWDWF |
| | SWLFLPSESKNHLHTTTQMAGTSSNTISAGWEPNEENEDERDRHLSFS |
| | GSGIDDDEDFISSTISTTPRAFDHTKQNQDWTQWNPSHSNPEVLLQTT |
| | TRMTDVDRNGTTAYEGNWNPEAHPPLIHHEHHEEEETPHSTSTIQATP |
| | SSTTEETATQKEQWFGNRWHEGYRQTPKEDSHSTTGTAAASAHTSHP |
| | MQGRTTPSPEDSSWTDFFNPISHPMGRGHQAGRRMDMDSSHSITLQP |
| | TANPNTGLVEDLDRTGPLSMTTQQSNSQSFSTSHEGLEEDKDHPTTST |
| | LTSSNRNDVTGGRRDPNHSEGSTTLLEGYTSHYPHTKESRTFIPVTSA |
| | KTGSFGVTAVTVGDSNSNVNRSLSGDQDTFHPSGGSHTTHGSESDGH |
| | SHGSQEGGANTTSGPIRTPQIPEWLIILASLLALALILAVCIAVNSRRS |
| SEQ ID NO: 11 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 10 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATNMDSSHSITLQPTANPNTGLVEDL |
| | DRTGPLSMTTQQSNSQSFSTSHEGLEEDKDHPTTSTLTSSNRNDVTG |
| | GRRDPNHSEGSTTLLEGYTSHYPHTKESRTFIPVTSAKTGSFGVTAVTV |
| | GDSNSNVNRSLSGDQDTFHPSGGSHTTHGSESDGHSHGSQEGGANT |
| | TSGPIRTPQIPEWLIILASLLALALILAVCIAVNSRRRCGQKKKLVINSGNG |
| | AVEDRKPSGLNGEASKSQEMVHLVNKESSETPDQFMTADETRNLQNV |
| | DMKIGV |
| SEQ ID NO: 12 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 11 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATNRNDVTGGRRDPNHSEGSTTLLE |
| | GYTSHYPHTKESRTFIPVTSAKTGSFGVTAVTVGDSNSNVNRSLSGDQ |
| | DTFHPSGGSHTTHGSESDGHSHGSQEGGANTTSGPIRTPQIPEWLIILA |
| | SLLALALILAVCIAVNSRRRCGQKKKLVINSGNGAVEDRKPSGLNGEAS |
| | KSQEMVHLVNKESSETPDQFMTADETRNLQNVDMKIGV |
| SEQ ID NO: 13 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 12 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATRDQDTFHPSGGSHTTHGSESDG |
| | HSHGSQEGGANTTSGPIRTPQIPEWLIILASLLALALILAVCIAVNSRRRC |
| | GQKKKLVINSGNGAVEDRKPSGLNGEASKSQEMVHLVNKESSETPDQ |
| | FMTADETRNLQNVDMKIGV |
| SEQ ID NO: 14 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 13 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATNMDSSHSITLQPTANPNTGLVEDL |
| | DRTGPLSMTTQQSNSQSFSTSHEGLEEDKDHPTTSTLTSSRDQDTFHP |
| | SGGSHTTHGSESDGHSHGSQEGGANTTSGPIRTPQIPEWLIILASLLAL |
| | ALILAVCIAVNSRRRCGQKKKLVINSGNGAVEDRKPSGLNGEASKSQE |
| | MVHLVNKESSETPDQFMTADETRNLQNVDMKIGV |
| SEQ ID NO: 15 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 14 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATNMDSSHSITLQPTANPNTGLVEDL |
| | DRTGPLSMTTRRDQDTFHPSGGSHTTHGSESDGHSHGSQEGGANTT |
| | SGPIRTPQIPEWLIILASLLALALILAVCIAVNSRRRCGQKKKLVINSGNGA |
| | VEDRKPSGLNGEASKSQEMVHLVNKESSETPDQFMTADETRNLQNVD |
| | MKIGV |
| SEQ ID NO: 16 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 15 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATRDQDTFHPSGGSHTTHGSESDG |
| | HSHGSQEGGANTTSGPIRTPQIPEWLIILASLLALALILAVCIAVNSRRS |
| SEQ ID NO: 17 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 16 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGALMS |
| | TSATATETATKRQETWDWFSWLFLPSESKNHLHTTTQMAGTSSNTISA |
| | GWEPNEENEDERDRHLSFSGSGIDDDEDFISSTISTTPRAFDHTKQNQ |
| | DWTQWNPSHSNPEVLLQTTTRMTDVDRNGTTAYEGNWNPEAHPPLIH |
| | HEHHEEEETPHSTSTTASAHTSHPMQGRTTPSPEDSSWTDFFNPISHP |
| | MGRGHQAGRRMDMDSSHSITLQPTANPNTGLVEDLDRTGPLSMTTQQ |
| | SNSQSFSTSHEGLEEDKDHPTTSTLTSSNRNDVTGGRRDPNHSEGST |
| | TLLEGYTSHYPHTKESRTFIPVTSAKTGSFGVTAVTVGDSNSNVNRSLS |
| | GDQDTFHPSGGSHTTHGSESDGHSHGSQEGGANTTSGPIRTPQIPEW |
| | LIILASLLALALILAVCIAVNSRRRCGQKKKLVINSGNGAVEDRKPSGLNG |
| | EASKSQEMVHLVNKESSETPDQFMTADETRNLQNVDMKIGV |
| SEQ ID NO: 18 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 17 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATTLMSTSATATETATKRQETWDWF |
| | SWLFLPSESKNHLHTTTQMAAGWEPNEENEDERDRHLSFSGSGIDDD |
| | EDFISSTISTTPRAFDHTKQNQDWTQWNPSHSNPEVLLQTTTRMTDVD |
| | RNGTTAYEGNWNPEAHPPLIHHEHHEEEETPHSTSTTASAHTSHPMQ |
| | GRTTPSPEDSSWTDFFNPISHPMGRGHQAGRRMDMDSSHSITLQPTA |
| | NPNTGLVEDLDRTGPLSMTTQQSNSQSFSTSHEGLEEDKDHPTTSTLT |
| | SSNRNDVTGGRRDPNHSEGSTTLLEGYTSHYPHTKESRTFIPVTSAKT |
| | GSFGVTAVTVGDSNSNVNRSLSGDQDTFHPSGGSHTTHGSESDGHS |
| | HGSQEGGANTTSGPIRTPQIPEWLIILASLLALALILAVCIAVNSRRRCGQ |
| | KKKLVINSGNGAVEDRKPSGLNGEASKSQEMVHLVNKESSETPDQFM |
| | TADETRNLQNVDMKIGV |
| SEQ ID NO: 19 | MDKFWWHAAWGLCLVPLSLAQIDLNITCRFAGVFHVEKNGRYSISRTE |
| Human CD44 | AADLCKAFNSTLPTMAQMEKALSIGFETCRYGFIEGHWIPRIHPNSICA |
| Isoform 18 | ANNTGVYILTSNTSQYDTYCFNASAPPEEDCTSVTDLPNAFDGPITITIV |
| | NRDGTRYVQKGEYRTNPEDIYPSNPTDDDVSSGSSSERSSTSGGYIFY |
| | TFSTVHPIPDEDSPWITDSTDRIPATRHSHGSQEGGANTTSGPIRTPQIP |
| | EWLIILASLLALALILAVCIAVNSRRRCGQKKKLVINSGNGAVEDRKPSGL |
| | NGEASKSQEMVHLVNKESSETPDQFMTADETRNLQNVDMKIGV |
| SEQ ID NO: 20 | MDKFWWHAAWGLCLVPLSLAQIDLNITCRFAGVFHVEKNGRYSISRTE |
| Human CD44 | AADLCKAFNSTLPTMAQMEKALSIGFETCSLHCSQQSKKVWAEEKASD |
| Isoform 19 | QQWQWSCGGQKAKWTQRRGQQVSGNGAFGEQGVVRNSRPVYDS |
| SEQ ID NO: 21 | GIDFSDYYYMC |
| CDR-H1 | |
| VR0851 | |
| SEQ ID NO: 22 | CIYGASSDSTYYASWAKG |
| CDR-H2 | |
| VR0851 | |
| SEQ ID NO: 23 | SDYLYDYAMNL |
| CDR-H3 | |
| VR0851 | |
| SEQ ID NO: 24 | QASQTINTYLS |
| CDR-L1 | |
| VR0851 | |
| SEQ ID NO: 25 | SASTLAS |
| CDR-L2 | |
| VR0851 | |
| SEQ ID NO: 26 | QSHYYIVSNGWP |
| CDR-L3 | |
| VR0851 | |
| SEQ ID NO: 27 | GFSLSSYAMS |
| CDR-H1 | |
| VR5479 | |
| SEQ ID NO: 28 | GIVSTGITYYASWAKG |
| CDR-H2 | |
| VR5479 | |
| SEQ ID NO: 29 | ILYSGGSGNYLHL |
| CDR-H3 | |
| VR5479 | |
| SEQ ID NO: 30 | QSSESVLSNNRFS |
| CDR-L1 | |
| VR5479 | |
| SEQ ID NO: 31 | GASSLAS |
| CDR-L2 | |
| VR5479 | |
| SEQ ID NO: 32 | AGYKSPTYDDWA |
| CDR-L3 | |
| VR5479 | |
| SEQ ID NO: 33 | QSLEESGGDLVKPGASLTLTCTASGIDFSDYYYMCWVRQAPGKGLELI |
| VH VR0851 | ACIYGASSDSTYYASWAKGRFTISKTSSTTVTLQMTSLTAADTATYFCV |
| | RSDYLYDYAMNLWGQGTLVTVSS |
| SEQ ID NO: 34 | AAGCTTCGAAGCCACCATGGAGACTGGGCTGCGCTGGCTTCTCCTG |
| VH nucl. | GTCGCTGTGCTCAAAGGTGTCCAGTGTCAGTCGTTGGAGGAGTCCG |
| VR0851 | GGGGAGATCTGGTCAAGCCTGGGGCATCCCTGACACTCACCTGCA |
| | CAGCCTCTGGAATCGACTTCAGTGACTACTACTACATGTGCTGGGT |
| | CCGCCAGGCTCCAGGGAAGGGGCTGGAGTTGATCGCCTGCATTTA |
| | TGGTGCTAGTAGTGATAGCACATACTACGCGAGCTGGGCGAAAGGC |
| | CGATTCACCATCTCCAAAACCTCGTCGACCACGGTGACTCTGCAAAT |
| | GACCAGTCTGACAGCCGCGGACACGGCCACCTATTTCTGTGTGAGA |
| | AGTGACTATCTATATGATTATGCCATGAACTTGTGGGGCCAAGGCAC |
| | CCTCGTCACCGTCTCGAGT |
| SEQ ID NO: 35 | DVVMTQTPASVEAAVGDIVTIKCQASQTINTYLSWYQQKPGQPPKLLIY |
| VL VR0851 | SASTLASGVPSRFKGSGSGTDYTLTISDLECADAATYYCQSHYYIVSNG |
| | WPFGGGTEVVVE |
| SEQ ID NO: 36 | AAGCTTCGAAGCCACCATGGACACGAGGGCCCCCACTCAGCTGCT |
| VL nucl. | GGGGCTCCTGCTGCTCTGGCTCCCAGGTGCCAGATGTGATGTTGT |
| VR0851 | GATGACCCAGACTCCAGCCTCCGTGGAGGCAGCTGTGGGAGACAT |
| | AGTCACCATCAAGTGCCAGGCCAGTCAGACCATTAATACTTACTTAT |
| | CCTGGTATCAGCAGAAACCAGGGCAGCCTCCCAAGCTCCTGATCTA |
| | TTCTGCATCCACTCTGGCATCTGGGGTCCCATCGCGGTTTAAAGGC |
| | AGTGGATCTGGGACAGATTACACTCTCACCATCAGCGACCTGGAGT |
| | GTGCCGATGCTGCCACTTACTACTGTCAAAGTCATTATTATATTGTTA |
| | GTAATGGTTGGCCTTTCGGCGGAGGGACCGAGGTGGTCGTCGAAC |
| | GTACG |
| SEQ ID NO: 37 | QSLEESGGRLVRPDETLTLTCTVSGFSLSSYAMSWVRQAPGKGLEYIG |
| VH VR5479 | GIVSTGITYYASWAKGRFTISKTSTTVTLKMTSLTTEDTATYFCVRILYS |
| | GGSGNYLHLWGQGTLVTVSS |
| SEQ ID NO: 38 | AAGCTTCGAAGCCACCATGGAGACTGGGCTGCGCTGGCTTCTCCTG |
| VH nucl. | GTCGCTGTGCTCAAAGGTGTCCAGTGTCAGTCGCTGGAGGAGTCC |
| VR5479 | GGGGGTCGCCTGGTCAGGCCTGACGAAACCCTGACACTCACCTGC |
| | ACAGTCTCTGGATTCTCCCTCAGTAGCTATGCAATGAGCTGGGTCC |
| | GCCAGGCTCCAGGGAAGGGGCTGGAATACATCGGAGGCATTGTTA |
| | GTACTGGTATCACATACTACGCGAGCTGGGCGAAAGGCCGATTCAC |
| | CATCTCCAAAACCTCGACCACGGTGACTCTGAAAATGACCAGTCTG |
| | ACAACCGAGGACACGGCCACCTATTTCTGTGTCAGAATATTATATAG |
| | TGGTGGTAGTGGTAATTACCTTCACTTGTGGGGCCAAGGCACCCTG |
| | GTCACCGTCTCGAGT |
| SEQ ID NO: 39 | ALVMTQTPSPVSAAVGGTVTISCQSSESVLSNNRFSWYQQKLGQPPKL |
| VL VR5479 | LIYGASSLASGVPSRFKGSGSGTEFTLTISGVECDDAATYYCAGYKSPT |
| | YDDWAFGGGTEVVVK |
| SEQ ID NO: 40 | AAGCTTCGAAGCCACCATGGACACGAGGGCCCCCACTCAGCTGCT |
| VL nucl. | GGGGCTCCTGCTGCTCTGGCTCCCAGGTGCCAGATGTGCGCTTGT |
| VR5479 | GATGACCCAGACTCCATCCCCTGTGTCTGCAGCTGTGGGAGGCACA |
| | GTCACCATCAGTTGCCAGTCCAGTGAGAGTGTTCTTAGTAATAACCG |
| | CTTTTCCTGGTATCAGCAGAAACTAGGGCAACCTCCCAAGCTCCTG |
| | ATCTATGGTGCATCCAGTCTGGCGTCTGGGGTCCCATCGCGGTTCA |
| | AAGGCAGTGGATCTGGGACAGAGTTCACTCTCACCATCAGTGGCGT |
| | GGAGTGTGACGATGCTGCCACTTACTACTGTGCAGGCTATAAAAGT |
| | CCTACCTATGATGACTGGGCTTTCGGCGGAGGGACCGAGGTGGTG |
| | GTCAAACGTACG |

Within the present invention, unless recited otherwise, human CTLA4 and human CD44 are always intended to be included in the term “CTLA4” or CD44”. However, unless “human CTLA4” and/or “human CD44” are explicitly used, the terms “CTLA4” and/or “CD44” include the same antigens in other species, especially non-primate (e.g. rodents) and non-human primate (such as cynomolgus monkey) species.

The present invention therefore provides for an antibody comprising a first antigen-binding portion binding human CTLA4 and a second antigen-binding portion binding human CD44. The first and the second antigen-binding portions are located on the same antibody, i.e. they are part of the same polypeptide chain, they associate via one or more non-covalent and/or covalent associations or are linked so as to form one single molecule.

The present invention also provides for an antibody comprising a first antigen-binding portion binding an extracellular domain region of human CTLA4 and a second antigen-binding portion binding an extracellular domain region of human CD44.

In particular, there is provided an antibody comprising a first antigen-binding portion binding human CTLA4 as defined in SEQ ID NO: 1 or from amino acid 1 to 223 of SEQ ID NO: 1 or from amino acid 36 to 233 of SEQ ID NO: 1 and a second antigen-binding portion binding human CD44 as defined in SEQ ID NO: 2 to 20 or variants or splicing isoforms generated by splicing out exons or alternative splicing within exons or binding the extracellular region of human CD44 as defined in SEQ ID NO: 2 to 20 or variants or splicing isoforms generated by splicing out exons or alternative splicing within exons. The first and the second antigen-binding portions are located on the same antibody, i.e. they are part of the same polypeptide chain, they associate via one or more non-covalent and/or covalent associations or are linked so as to form one single molecule.

Ipilimumab (Yervoy™) is an anti-CTLA4 fully human IgG1 monoclonal antibody currently approved for the treatment of cancer and tremelimumab is an anti-CTLA4 fully human IgG2 monoclonal antibody in development. In one embodiment, the antibody comprises a first antigen-binding portion binding human CTLA4, which is the antigen-binding portion of Ipilimumab, and a second antigen-binding portion binding human CD44. In another embodiment, the antibody comprises a first antigen-binding portion binding human CTLA4, which is the antigen-binding portion of tremelimumab, and a second antigen-binding portion binding human CD44.

The monoclonal antibody of the present invention, upon binding of CTLA4 and CD44, stimulates T cell activation, i.e. enhancement of cytokine production; further activates T cells and enhances induction of T cell proliferation, and in particular, the monoclonal antibody comprising a first antigen-binding portion binding CTLA4 and a second antigen-binding portion binding CD44 enhances cytokine production and enhances induction of T cell proliferation in the presence of Staphylococcus aureus Enterotoxin B (SEB) stimulation. More specifically, the monoclonal antibody comprising a first antigen-binding portion binding CTLA4 and a second antigen-binding portion binding CD44 enhances cytokine production and enhances induction of T cell proliferation in the presence of SEB stimulation but it does not activate unstimulated T cells. More specifically, the T cell is at least a CD4+ T cell or at least a CD8+ T cell or a mixture thereof.

The term “activate” (and grammatical variations thereof) as used herein at least includes the upregulation of specific cytokines, i.e. increased transcription and/or translation of these cytokines and/or release/secretion of these cytokines.

Hence, the present invention provides for a monoclonal antibody comprising a first antigen-binding portion binding CTLA4 and a second antigen-binding portion binding CD44 capable of activating T cells in the presence of an anti-CD3 stimulation wherein the further activation of T cell is measured as an upregulation of cytokines production and/or an enhancement of T cell proliferation.

Upregulation or enhancement of cytokine production includes but is not limited to the upregulation of Interleukin 2 (IL-2).

In one preferred embodiment of the present invention, the monoclonal antibody comprising a first antigen-binding portion binding CTLA4 and a second antigen-binding portion binding CD44 is capable of upregulating or enhancing cytokine production and/or enhancing T cell proliferation in the presence of SEB stimulation wherein upregulating or enhancing cytokine production results in an upregulation of IL-2.

The term “antibody” as used herein includes whole immunoglobulin molecules and antigen-binding portions of immunoglobulin molecules associated via non-covalent and/or covalent associations or linked together, optionally via a linker.

In one embodiment, the antigen-binding portions binding CTLA4 and CD44 are the antigen-binding portions of an IgG, wherein one arm binds CTLA4 and the other arm binds CD44.

In another embodiment, the antigen-binding portions comprised in the antibody are functionally active fragments or derivatives of a whole immunoglobulin and may be, but are not limited to, VH, VL, VHH, Fv, scFv fragment (including dsscFv), Fab fragments, modified Fab fragments, Fab′ fragments, F(ab′)₂ fragments, Fv and epitope-binding fragments of any of the above.

Other antibody fragments include those described in WO2005003169, WO2005003170, WO2005003171, WO2009040562 and WO2010035012. Functionally active fragments or derivative of a whole immunoglobulin and methods of producing them are well known in the art, see for example Verma et al., 1998, Journal of Immunological Methods, 216, 165-181; Adair and Lawson, 2005. Therapeutic antibodies. Drug Design Reviews— Online 2(3):209-217.

In one embodiment of the invention each of the antigen-binding portions are independently selected from a Fab, a Fab′, a scFv or a VHH. In one embodiment, the antigen-binding portion binding CTLA4 is a Fab whilst the antigen-binding portion binding CD44 is a scFv. In another embodiment, the antigen-binding portion binding CD44 is a Fab whilst the antigen-binding portion binding CTLA4 is a scFv. In another embodiment, both antigen-binding portions are a Fab or scFv.

In one preferred embodiment, the antibody is monoclonal, which means that the antigen-binding portions comprised therein are all monoclonal. Therefore, in one preferred embodiment of the present invention, there is provided a monoclonal antibody comprising a first antigen-binding portion binding CTLA4 and a second antigen-binding portion binding CD44. Preferably, this antibody is capable of upregulating or enhancing cytokine production and/or enhancing induction of T cell proliferation in the presence of SEB stimulation wherein upregulating or enhancing cytokine production results in an upregulation of IL-2.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J. et al, 1996, Proc. Natl. Acad. Sci. USA 93(15):7843-78481; WO92/02551; WO2004/051268 and WO2004/106377.

The antibodies of the present invention can also be generated using various phage display methods known in the art and include those disclosed by Brinkman et al. (in J. Immunol. Methods, 1995, 182: 41-50), Ames et al. (J. Immunol. Methods, 1995, 184: 177-186), Kettleborough et al. (Eur. J. Immunol. 1994, 24:952-958), Persic et al. (Gene, 1997 187 9-18), Burton et al. (Advances in Immunology, 1994, 57: 191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. When the antigen-binding portions comprised in the antibody are functionally active fragments or derivatives of a whole immunoglobulin such as single chain antibodies, they may be made such as those described in U.S. Pat. No. 4,946,778 which can also be adapted to produce single chain antibodies binding to CTLA4 and CD44. Transgenic mice, or other organisms, including other mammals, may be used to express antibodies, including those within the scope of the invention.

The antibody of the present invention may be chimeric, human or humanised.

Chimeric antibodies are those antibodies encoded by immunoglobulin genes that have been genetically engineered so that the light and heavy chain genes are composed of immunoglobulin gene segments belonging to different species.

Humanized, antibodies are antibody molecules from non-human species having one or more complementarity determining regions (CDRs) from the non-human species and a framework region from a human immunoglobulin molecule (see, e.g. U.S. Pat. No. 5,585,089; WO91/09967). Preferably the antibody of the present invention is humanized. In one embodiment of the present invention, there is provided an antibody, preferably a monoclonal antibody, comprising a first antigen-binding portion binding CTLA4 and a second antigen-binding portion binding CD44, wherein the antibody is capable of upregulating or enhancing cytokine production and/or enhancing induction of T cell proliferation in the presence of SEB stimulation wherein upregulating or enhancing cytokine production results in an upregulation of IL-2.

In humanized antibodies, the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment, rather than the entire CDR being transferred, only one or more of the specificity determining residues from any one of the CDRs described herein above are transferred to the human antibody framework (see, for example, Kashmiri et al., 2005, Methods, 36, 25-34). In one embodiment, only the specificity determining residues from one or more of the CDRs described herein above are transferred to the human antibody framework. When the CDRs or specificity determining residues are grafted, any appropriate acceptor variable region framework sequence may be used having regard to the class/type of the donor antibody from which the CDRs are derived, including mouse, primate and human framework regions. Preferably, the humanized antibody according to the invention comprises a variable domain comprising human acceptor framework regions as well as one or more of the CDRs or specificity determining residues described above. Thus, provided in one embodiment is a humanized monoclonal antibody comprising an antigen-binding portion binding CTLA4 and an antigen-binding portion binding CD44, wherein each antigen-binding portion comprises a variable domain comprising human acceptor framework regions and non-human donor CDRs.

Examples of human frameworks which can be used in the invention are KOL, NEWM, REI, EU, TUR, TEI, LAY and POM (Kabat et al, supra). For example, KOL and NEWM can be used for the heavy chain, REI can be used for the light chain and EU, LAY and POM can be used for both the heavy chain and the light chain. Alternatively, human germline sequences may be used; these are available at, for example: http://vbase.mrc-cpe.cam.ac.uk/. In a CDR-grafted antibody of the invention, the acceptor heavy and light chains do not necessarily need to be derived from the same antibody and may, if desired, comprise composite chains having framework regions derived from different chains.

Fully human antibodies are those antibodies in which the variable regions and the constant regions (where present) of both the heavy and the light chains are all of human origin, or substantially identical to sequences of human origin, not necessarily from the same antibody. Examples of fully human antibodies may include antibodies produced for example by the phage display methods described above and antibodies produced by mice in which the murine immunoglobulin variable and constant region genes have been replaced by their human counterparts, e.g., as described in general terms in EP0546073, U.S. Pat. Nos. 5,545,806, 5,569,825, 5,625,126, 5,633,425, 5,661,016, 5,770,429, EP 0438474 and EP0463151.

Furthermore, the antibody of the invention may comprise a heavy chain constant region selected from an IgG1, an IgG2, an IgG3 or an IgG4 isotype, or a variant thereof. The constant region domains of the antibody of the invention, if present, may be selected having regard to the proposed function of the antibody, and in particular the effector functions which may be required. For example, the human IgG constant region domains of the IgG1 and IgG3 isotypes may be used when the antibody effector functions are required. Alternatively, IgG2 and IgG4 isotypes may be used when the antibody effector functions are not required. For example, IgG4 molecules in which the serine at position 241 has been changed to proline as described in Angal et al., Molecular Immunology, 1993, 30 (1), 105-108 may be used. Particularly preferred is the IgG4 constant domain that comprises this change.

It should also be appreciated that antigen-binding portions comprised in the antibody of the invention such as the functionally-active fragments or derivatives of a whole immunoglobulin fragments described above, may be incorporated into other antibody formats than being the antigen-binding portions of the classic IgG format. Alternative format to the classic IgG may include those known in the art and those described herein, such as DVD-Igs, FabFvs for example as disclosed in WO2009/040562 and WO2010/035012, diabodies, triabodies, tetrabodies etc. Other examples include a diabody, triabody, tetrabody, bibodies and tribodies (see for example Holliger and Hudson, 2005, Nature Biotech 23(9): 1 126-1136; Schoonjans et al. 2001, Biomolecular Engineering, 17 (6), 193-202), tandem scFv, tandem scFv-Fc, FabFv, Fab′Fv, FabdsFv, Fab-scFv, Fab′-scFv, diFab, diFab′, scdiabody, scdiabody-Fc, ScFv-Fc-scFv, scdiabody-CH3, IgG-scFv, scFv-IgG, V-IgG, IgG-V, DVD-Ig, DuoBody, Fab-Fv-Fv, Fab-Fv-Fc and Fab-dsFv-PEG fragments described in WO2009040562, WO2010035012, WO2011/08609, WO2011/030107 and WO2011/061492, respectively.

Furthermore, the antibody of the invention may comprise along with the antigen-binding portions binding CTLA4 and CD44, also at least an additional antigen-binding portion. Therefore, in one embodiment, there is provided an antibody, preferably a monoclonal antibody, comprising a first antigen-binding portion binding CTLA4 and a second antigen-binding portion binding CD44, wherein the antibody is humanised and wherein the antibody further comprises an additional antigen-binding portion. More preferably this antibody is capable of upregulating or enhancing cytokine production and/or enhancing induction of T cell proliferation. The stimulation may be SEB stimulation wherein upregulating or enhancing cytokine production results in an upregulation of IL-2 release.

In one embodiment, the additional antigen-binding portion is capable of increasing, i.e. extending, the half-life of the antibody. Preferably, the additional antigen-binding portion binds albumin, more preferably human serum albumin.

In one embodiment, the first antigen-binding portion binding CTLA4 of the antibody according to the present invention comprises a first heavy chain variable region and a first light chain variable region and the second antigen-binding portion binding CD44 comprises a second heavy chain variable region and a second light chain variable region and wherein:
a. The first heavy chain variable region comprises a CDR-H1 comprising SEQ ID NO: 21, a CDR-H2 comprising SEQ ID NO: 22 and a CDR-H3 comprising SEQ ID NO: 23; and
b. The first light chain variable region comprises a CDR-L1 comprising SEQ ID NO: 24, a CDR-L2 comprising SEQ ID NO: 25 and a CDR-L3 comprising SEQ ID NO: 26; and
c. The second heavy chain variable region comprises a CDR-H1 comprising SEQ ID NO: 27, a CDR-H2 comprising SEQ ID NO: 28 and a CDR-H3 comprising SEQ ID NO: 29; and
d. The second light chain variable region comprises a CDR-L1 comprising SEQ ID NO: 30, a CDR-L2 comprising SEQ ID NO: 31 and a CDR-L3 comprising SEQ ID NO: 32;
or
e. The first heavy chain variable region comprises SEQ ID NO: 33 and the first light chain variable region comprises SEQ ID NO: 35; and the second heavy chain variable region comprises SEQ ID NO: 37 and second light chain variable region comprises SEQ ID NO: 39;
   or
f. The first heavy chain variable region is encoded by a nucleotide sequence comprising SEQ ID NO: 34 and the first light chain variable region is encoded by a nucleotide sequence comprising SEQ ID NO: 36; and the second heavy chain variable region is encoded by a nucleotide sequence comprising SEQ ID NO: 38 and second light chain variable region is encoded by a nucleotide sequence comprising SEQ ID NO: 40.

In one embodiment, the antibody according to the present invention is prepared according to the disclosure of WO2015/181282, WO2016/009030, WO2016/009029, WO2017/093402, WO2017/093404 and WO2017/093406, which are all incorporated herein by reference.

More specifically, the antibody is made by the heterodimerization of a Fab-X and a Fab-Y.

Fab-X comprises a Fab fragment which comprises the first antigen-binding portion binding CTLA4 which comprises a first heavy chain variable region and a first light chain variable region wherein the first heavy chain variable region comprises a CDR-H1 comprising SEQ ID NO: 21, a CDR-H2 comprising SEQ ID NO: 22 and a CDR-H3 comprising SEQ ID NO: 23; and the first light chain variable region comprises a CDR-L1 comprising SEQ ID NO: 24, a CDR-L2 comprising SEQ ID NO: 25 and a CDR-L3 comprising SEQ ID NO: 26. The Fab comprising the first antigen-binding portion binding CTLA4 is linked to a scFv (clone 52SR4), preferably via a peptide linker to the C-terminal of the CH1 domain of the Fab fragment and the VL domain of the scFv. The scFv may itself also contains a peptide linker located in between its VL and VH domains.

Fab-Y also comprises a Fab fragment which comprises the second antigen-binding portion binding CD44 which comprises a second heavy chain variable region and a second light chain variable region and wherein the second heavy chain variable region comprises a CDR-H1 comprising SEQ ID NO: 27, a CDR-H2 comprising SEQ ID NO: 28 and a CDR-H3 comprising SEQ ID NO: 29; and the second light chain variable region comprises a CDR-L1 comprising SEQ ID NO: 30, a CDR-L2 comprising SEQ ID NO: 31 and a CDR-L3 comprising SEQ ID NO: 32. The Fab comprising the second antigen-binding portion binding CD44 is linked to a peptide GCN4 (clone 7P14P), preferably via a peptide linker to the CH1 domain of the Fab fragment.

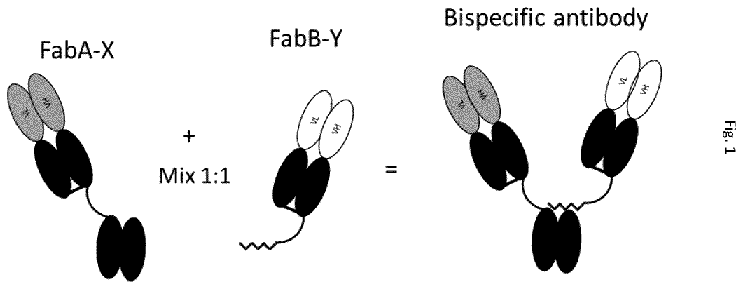

The scFv of Fab-X is specific for and complementary to the peptide GCN4 of Fab-Y. As a result, when the Fab-X and the Fab-Y are brought into contact with each other, a non-covalent binding interaction between the scFv and GCN4 peptide occurs, thereby physically retaining the two antigen-binding portions in the form of a complex resulting in an antibody comprising two antigen-binding portions on the same molecule (FIG. 1).

In another embodiment, the Fab-X comprises the first antigen-binding portion binding CTLA4 which comprises a first heavy chain variable region comprising SEQ ID NO: 33 and the first light chain variable region comprising SEQ ID NO: 35; and the Fab-Y comprises the second antigen-binding portion binding CD44 which comprises the second heavy chain variable region comprising SEQ ID NO: 37 and second light chain variable region comprising SEQ ID NO: 39.

Binding specificities may be exchanged between Fab-X and Fab-Y, i.e. in one embodiment Fab-X may comprise the antigen-binding portion binding to CTLA4 and Fab-Y the antigen-binding portion binding to CD44; in another embodiment, Fab-Y may comprise the antigen-binding portion binding to CTLA4 and Fab-X the antigen-binding portion binding to CD44.

The antibody of the present invention may be comprised in a pharmaceutical composition along with one or more pharmaceutically acceptable excipients. By pharmaceutical composition is intended a composition for both therapeutic and diagnostic use. In another aspect, the present invention provides for a pharmaceutical composition comprising an antibody, preferably a monoclonal antibody, comprising a first antigen-binding portion binding CTLA4 and a second antigen-binding portion binding CD44, wherein the antibody is preferably humanised and wherein the composition comprises one or more pharmaceutically acceptable excipients.

Pharmaceutically acceptable excipients in therapeutic compositions may additionally contain liquids such as water, saline, glycerol and ethanol. Additionally, auxiliary substances, such as wetting or emulsifying agents or pH buffering substances, may be present in such compositions. Such excipients enable the pharmaceutical compositions to be formulated as tablets, pills, dragees, capsules, liquids, gels, syrups, slurries and suspensions, for ingestion by the patient.

The antibody of the present invention and the pharmaceutical composition comprising such antibody may be used in therapy.

Therefore, in another aspect, the present invention provides for an antibody, preferably a monoclonal antibody, or a pharmaceutical composition comprising the antibody, and one or more pharmaceutically acceptable excipients, wherein the antibody comprises a first antigen-binding portion binding CTLA4 and a second antigen-binding portion binding CD44, wherein the antibody is preferably humanised and is for use in therapy.

In another aspect, the present invention provides for an antibody, preferably a monoclonal antibody, or a pharmaceutical composition comprising the antibody and one or more pharmaceutically acceptable excipients, wherein the antibody comprises a first antigen-binding portion binding CTLA4 and a second antigen-binding portion binding CD44, wherein the antibody is preferably humanised and is for use in the treatment of cancer and/or an infectious disease.

The bispecific antibody according to the present invention has shown, unlike monospecific anti-CTLA antibodies such as ipilimumab, to be able to stimulate T cell activation in the presence of regulatory T cells, eventually leading to reversal of immune suppression, at least in part or in full, in the cancer microenvironment.

In one embodiment of the present invention, there is provided for an antibody, preferably a monoclonal antibody, or a pharmaceutical composition comprising the antibody and one or more pharmaceutically acceptable excipients, wherein the antibody comprises a first antigen-binding portion binding CTLA4 and a second antigen-binding portion binding CD44, wherein the antibody is preferably humanised and is for use in the treatment of cancer and/or an infectious disease, wherein the antibody stimulates T cell activation in the presence of regulatory T cells, reversing immune suppression, at least in part or in full.

In yet another aspect, the present invention provides for a method for treating a subject afflicted with cancer and/or an infectious disease, comprising administering to the subject a pharmaceutically effective amount of an antibody, preferably a monoclonal antibody, or a pharmaceutical composition comprising the antibody, and one or more pharmaceutically acceptable excipients, wherein the antibody comprises a first antigen-binding portion binding CTLA4 and a second antigen-binding portion binding CD44, wherein the antibody is preferably humanised.

The subject to be treated is preferably a human subject. In one embodiment, there is provided for a method for treating a human subject afflicted with cancer and/or an infectious disease, comprising administering to the subject a pharmaceutically effective amount of an antibody, preferably a monoclonal antibody, or a pharmaceutical composition comprising the antibody and one or more pharmaceutically acceptable excipients, wherein the antibody comprises a first antigen-binding portion binding CTLA4 and a second antigen-binding portion binding CD44, wherein the antibody is preferably humanised.

In another embodiment, there is provided a method for treating a human subject afflicted with cancer and/or an infectious disease, comprising administering to the subject a pharmaceutically effective amount of an antibody, preferably a monoclonal antibody, or a pharmaceutical composition comprising the antibody and one or more pharmaceutically acceptable excipients, wherein the antibody comprises a first antigen-binding portion binding CTLA4 and a second antigen-binding portion binding CD44, wherein the antibody is preferably humanised and is for use in the treatment of cancer and/or an infectious disease, wherein the antibody stimulates T cell activation in the present of regulatory T cells, reversing immune suppression, at least in part or in full.

In another aspect of the present invention, there is provided the use of an antibody, preferably a monoclonal antibody, or a pharmaceutical composition comprising the antibody, and one or more pharmaceutically acceptable excipients, wherein the antibody comprises a first antigen-binding portion binding CTLA4 and a second antigen-binding portion binding CD44, wherein the antibody is preferably humanised, in the manufacture of a medicament for treating cancer and/or an infectious disease.

Example of cancers that may be treated using the antibody, or pharmaceutical composition comprising such antibody, include but are not limited to, Acute Lymphoblastic Leukaemia, Acute Myeloid Leukaemia, Adrenocortical Carcinoma, AIDS-Related Cancers Kaposi Sarcoma (Soft Tissue Sarcoma), AIDS-Related Lymphoma, Primary CNS Lymphoma, Anal Cancer, Appendix Cancer Astrocytomas, Atypical Teratoid/Rhabdoid Tumour, Brain Cancer, Basal Cell Carcinoma of the Skin, Bile Duct Cancer, Bladder Cancer, Bone Cancer (includes Ewing Sarcoma and Osteosarcoma and Malignant Fibrous Histiocytoma), Breast Cancer, Bronchial Tumours, Burkitt Lymphoma, Carcinoid Tumour, Cardiac (Heart) Tumours, Embryonal Tumours, Germ Cell Tumour, Primary CNS Lymphoma, Cervical Cancer, Cholangiocarcinoma, Chordoma, Chronic Lymphocytic Leukaemia, Chronic Myelogenous Leukaemia, Chronic Myeloproliferative Neoplasms, Colorectal Cancer, Craniopharyngioma, Cutaneous T-Cell Lymphoma, Ductal Carcinoma In Situ, Endometrial Cancer (Uterine Cancer), Ependymoma, Oesophageal Cancer, Esthesioneuroblastoma (Head and Neck Cancer), Ewing Sarcoma (Bone Cancer), Extracranial Germ Cell Tumour, Extragonadal Germ Cell Tumour, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumour, Gastrointestinal Stromal Tumours (Soft Tissue Sarcoma), Ovarian Germ Cell Tumours, Testicular Cancer, Gestational Trophoblastic Disease, Hairy Cell Leukaemia, Head and Neck Cancer, Hepatocellular (Liver) Cancer, Histiocytosis, Langerhans Cell Hodgkin Lymphoma, Hypopharyngeal Cancer, Intraocular Melanoma, Islet Cell Tumours, Pancreatic Neuroendocrine Tumours, Kaposi Sarcoma (Soft Tissue Sarcoma), Kidney (Renal Cell) Cancer, Langerhans Cell Histiocytosis, Laryngeal Cancer, Leukaemia, Lip and Oral Cavity Cancer, Liver Cancer, Lung Cancer (Non-Small Cell and Small Cell), Male Breast Cancer, Malignant Fibrous Histiocytoma of Bone and Osteosarcoma, Melanoma, Intraocular (Eye) Childhood Intraocular, Merkel Cell Carcinoma, Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukaemia, Chronic, Myeloid Leukaemia, Acute, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumours (Islet Cell Tumours), Papillomatosis, Paraganglioma, Paranasal Sinus and Nasal Cavity Cancer, Parathyroid Cancer, Penile Cancer, Pharyngeal Cancer, Pheochromocytoma, Pituitary Tumour, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma Childhood Rhabdomyosarcoma, Childhood Vascular Tumours, Ewing Sarcoma, Kaposi Sarcoma, Osteosarcoma, Soft Tissue Sarcoma, Uterine Sarcoma, Sézary Syndrome, Skin Cancer, Small Intestine Cancer, Squamous Cell Carcinoma of the Skin, Squamous Neck Cancer with Occult Primary, Stomach (Gastric) Cancer, Cutaneous T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Urethral Cancer, Uterine Cancer, Endometrial Uterine Sarcoma, Vaginal Cancer, Vascular Tumours, Vulvar Cancer and Wilms Tumour, and any combinations of these cancers. The present invention is also applicable to treatment of metastatic cancers.

The antibody according to the present invention, or the pharmaceutical composition comprising such antibody, may be administered concomitantly or sequentially to one or more additional cancer therapies. By cancer therapies is intended drug based therapies as well as other type of cancer therapies such as radiotherapies.

The invention will now be further described by way of examples with references to embodiments illustrated in the accompanying drawings

### EXAMPLES

### Example 1: T Cell Activation Primary Screen

The activation status of T cells can be assessed through their expression of cell surface markers and secreted cytokines which play important roles in cellular function. Activated T cells produce increased amounts of granzyme B, IFNgamma and IL-2, essential mediators to effect killing of virus-infected or tumour cell targets. T cells in the tumour microenvironment are often maintained in a suppressed state and express only low levels of these proteins. Agents that overcome this suppression and induce T cell activation, proliferation and cytotoxic activity have tremendous therapeutic potential as they may unleash effective anti-tumour T cell responses and promote cancer elimination.

To identify novel modulators of T cell activation, a large screen was undertaken whereby 49 antigen specificities were combined to generate a grid of bispecific antibodies with a theoretical size of 1,176 possible bispecific combinations. The specificities were selected from the literature as being expressed on T cells or expressed on other cell types involved in interactions with T cells. Of this potential grid, 969 novel antigen bispecific constructs were tested, covering 82.4% of the possible combinations. Between 1 and 4 different antibodies were tested for each specificity, and all were tested on peripheral blood mononuclear cells (PBMC) from donors in combination with a negative control arm to identify the effect of the monovalent forms of the construct.

PBMC represent the major leukocyte classes involved in both innate and adaptive immunity, apart from granulocytes. PBMC comprise a heterogenous population of cells which when manipulated in vitro provide a relatively more relevant physiological environment compared to isolated component cell types such as T cells and monocytes, that are no longer capable of responding to paracrine and autocrine signals provided by other cells. As such identification of molecules modulating specific subsets of cells within the wider PBMC population, have increased translational potential to more complex biological systems, ultimately increasing success rates for modulating immune cell interactions in disease.

Each bispecific combination was tested on two PBMC donors. This negative control arm is a Fab from an antibody raised to an antigen not expressed on PBMC.

Fusion proteins were prepared according to the disclosure of WO2015/181282, WO2016/009030, WO2016/009029, WO2017/093402, WO2017/093404 and WO2017/093406, which are all incorporated herein by reference. The first fusion protein (A-X) includes a Fab fragment (A of the A-X) with specificity to one antigen, which is linked to X, a scFv (clone 52SR4) via a peptide linker to the C-terminal of the CH1 domain of the Fab fragment and the VL domain of the scFv. The scFv itself also contains a peptide linker located in between its VL and VH domains.

The second fusion protein (B-Y) includes a Fab fragment (B of the B-Y) with specificity to another antigen. However, in comparison to the first protein, the Fab fragment B is attached to Y, a peptide GCN4 (clone 7P14P) via a peptide linker to the CH1 domain of the Fab fragment.

The scFv, X, is specific for and complementary to the peptide GCN4, Y. As a result, when the two fusion proteins are brought into contact with each other, a non-covalent binding interaction between the scFv and GCN4 peptide occurs, thereby physically retaining the two fusion proteins in the form of a complex mimicking an antibody comprising antigen-binding portion on the same molecule (FIG. 1).

Purified Fab-X and Fab-Y with varying specificities were incubated together for 60 minutes (in a 37° C., 5% CO₂ environment) at an equimolar concentration. The final molarity of each tested complex was 100 nM. In 384-well tissue culture plates, 1.0×10⁵ PBMC were added to wells, to which were added pre-formed Fab-X/Fab-Y bispecific antibodies. Following the bispecific antibodies addition, the cells were incubated for 48 hours at 37° C., 5% CO₂, with or without 1 μg/mL (final concentration) Staphylococcal Enterotoxin-B (SEB) superantigen (SAg) or 250 ng/mL (final concentration) anti-human CD3 antibody (clone UCHT1). After 48 hours the plates were centrifuged at 500×g for 5 minutes at 4° C. Cell culture conditioned media was transferred from the cell pellets to fresh plates and frozen at −80° C.

The collected conditioned media were thawed and diluted 20-fold for the anti-CD3 and stimulated plates, and 5-fold for the unstimulated plates. The diluted conditioned media were then assayed for levels of the protein IL-2 using an IntelliCyt® QBead PlexScreen.

When considering antigen pairs capable of upregulating the levels of IL-2 in the conditioned media in conjunction with SEB, but not anti-CD3 and unstimulated condition, CD44-CTLA4 pair was identified.

CTLA4-CD44 pair was therefore taken into subsequent assays to show that its effect was repeatable across a larger number of donors.

### Example 2: CTLA4-CD44 Follow Up Assay

To confirm the effect of CD44-CTLA4 on increased IL-2 secretion, a further 4 PBMC donors were assayed in SEB, anti-CD3 and unstimulated conditions.

A grid of fusion proteins Fab-X and Fab-Y were created by diluting equimolar (1 μM) quantities of Fab-X (Fab-scFv) and Fab-Y (Fab-peptide) with specificity for CD44 and CTLA4 in TexMACS™ media (Miltenyi Biotec®) containing 100 U/mL penicillin/100 μg/mL streptomycin. Mixtures of equimolar (1 μM) Fab-Y proteins were also generated in the same manner. The Fab-X and Fab-Y fusion proteins were incubated together for 1 hour (in a 37° C./5% CO₂ environment), at a final concentration of 500 nM. Negative control wells contained TexMACS™ media only were also generated alongside the Fab-X and Fab-Y wells.

During this time, cryopreserved human PBMC isolated from platelet leukapheresis cones were thawed and washed in TexMACS™ media and resuspended at 3.33×10⁶ cells/mL. The PBMC were then seeded into 384-well flat bottom tissue culture plates (Greiner Bio-One®) at 30 μL/well (1.0×10⁵ PBMC). A total of 10 μL of Fab-X/Fab-Y bispecific antibodies were transferred to the plates containing 30 μL PBMC. The PBMC were then either left unstimulated by the addition of 10 μL of TexMACS™ media, or stimulated with 10 μL of SEB (1 μg/mL final concentration). This resulted in a final assay concentration of Fab-X/Fab-Y bispecific antibodies of 100 nM. The plates were then returned to a 37° C./5% CO₂ environment for 48 hours.

After 48 hours the plates were centrifuged at 500×g for 5 minutes at 4° C. Cell culture conditioned media was transferred from the cell pellets to fresh plates and frozen at −80° C. On the day of analysis, the conditioned media were thawed and diluted 20-fold. The diluted conditioned media were then assayed for levels of IL-2 using an IntelliCyt® QBead PlexScreen.

The data analysis software package ForeCyt™ (IntelliCyt®) was used to measure the MFI values for the IL-2 detection beads. The data were then used to calculate the Log 2 fold changes of IL-2 levels relative to control well values.

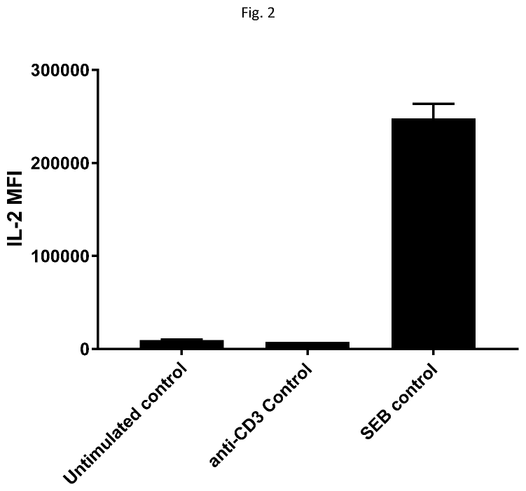

IL-2 could not be detected in either the unstimulated or anti-CD3 stimulated control wells, while large increases in MFI and therefore protein levels were detected in SEB stimulated control samples (FIG. 2).

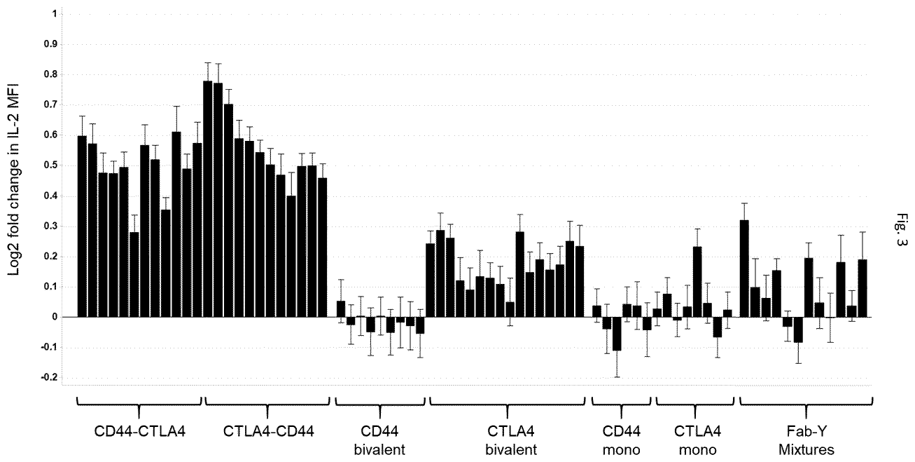

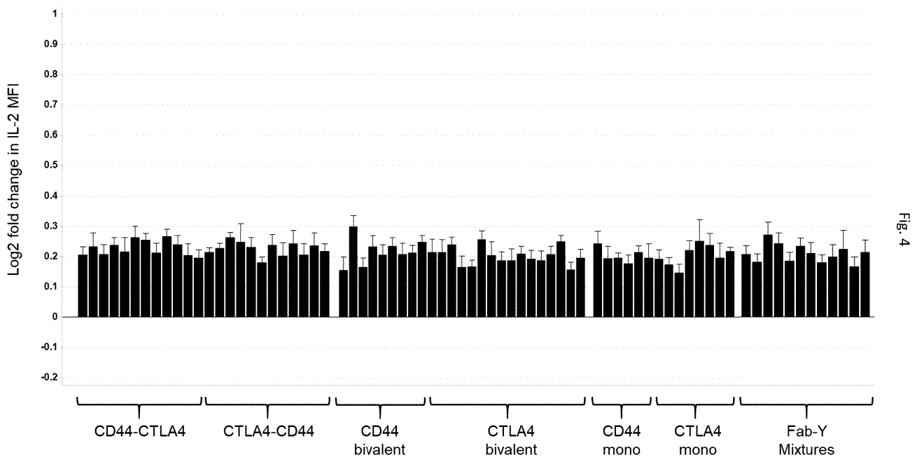

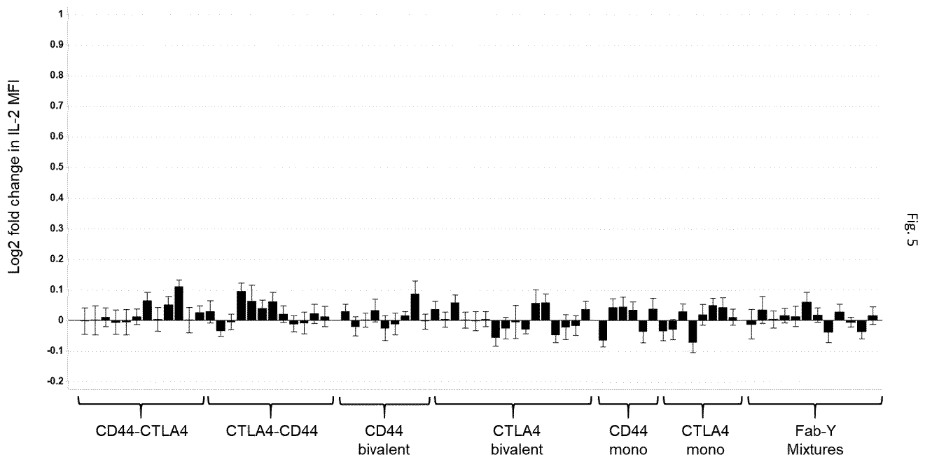

Twenty-four Fab-X and Fab-Y complexes showed increased levels of secreted IL-2 to varying degrees when PBMC were stimulated with SEB (FIG. 3), while the control constructs did not lead to this increase. These combinations however did not increase the level of IL-2 in anti-CD3 stimulation or unstimulated conditions (FIGS. 4 and 5).

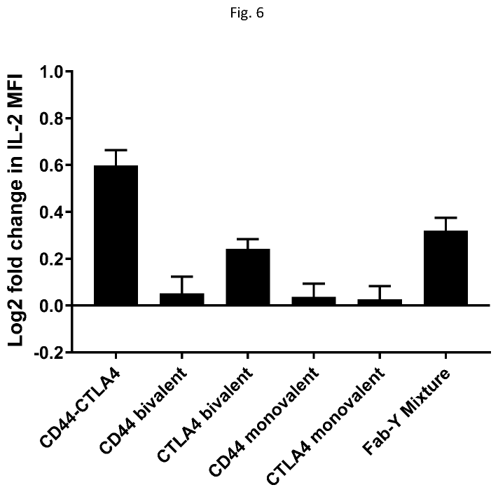

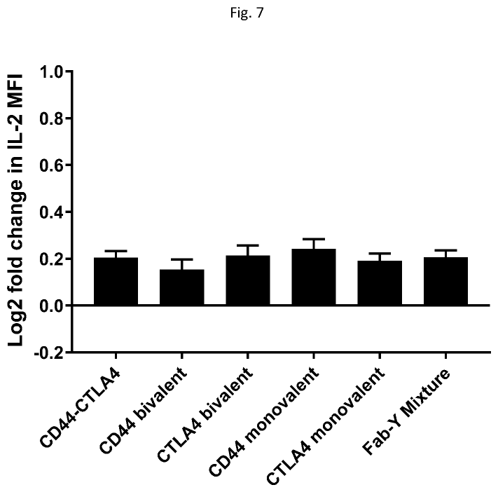

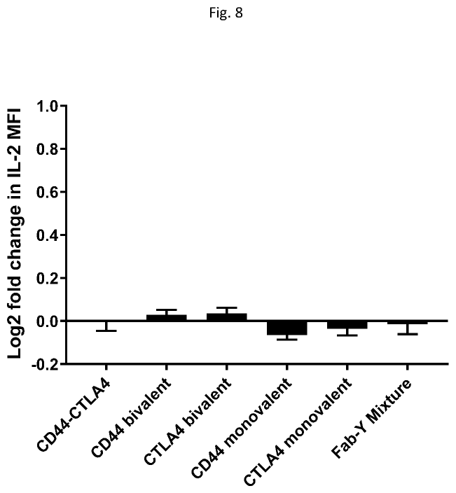

FIGS. 6 to 8 show a representative CD44-CTLA4 bispecific antibody as well as the bivalent, monovalent and Fab-Y mixture controls specific for this combination. The CD44-CTLA4 bispecific antibody is shown to increase the levels of secreted IL-2 when added to PBMC stimulated with SEB for 48 hours (FIG. 6). The bivalents (i.e. formed by a fusion where both Fab in the Fab-X and Fab-Y are specific for CD44 as the case may be) and monovalent antibodies for CD44 (i.e. formed by a fusion where one Fab is specific for CD44 but the other component Fab is a negative control) did not lead to a similar increase in the IL-2, suggesting that the binding of CD44 alone cannot induce IL-2 secretion in the absence of CTLA4. Some small increases in IL-2 secretion could be detected by the CTLA4 bivalent controls, however these were small increases, which were greatly enhanced by the coupling with CD44. Furthermore, the mixture of Fab-Y binding CD44 and Fab-Y binding CTLA4 had no enhanced stimulatory effect on the T cell populations compared to the CTLA4 controls, implying the requirement for the antigen-binding portions binding CD44 and CTLA4 to be on the same chain, associated via non-covalent associations or linked for the stimulatory function to occur. When studied in anti-CD3 or unstimulated conditions the CD44-CTLA4 bispecific antibodies did not lead to any increases in IL-2 secretion (FIG. 7-8).

### Example 3: Comparison of the Ability of CD44-CTLA4 Bispecific Antibody to Activate T Cells Versus Ipilimumab and Nivolumab

Ipilimumab (an anti-CTLA-4 antibody) was the first checkpoint inhibitor to be approved in 2011 as a treatment for melanoma, closely followed by FDA approval of anti-PD1 directed antibodies, pembrolizumab and nivolumab in 2014 (Hargadon et al., International Immunopharmacol. 62:29-39 (2018)). Whilst there are still significant challenges in understanding differences in efficacy across patient groups, ranging from complete responses, to treatment relapse and even failure to respond, (Haslam and Prasad. JAMA Network Open.5:2e192535 (2019)), these molecules represent current clinically-validated references for immunotherapy in a range of cancer types and have been utilised in the present studies for benchmarking the activity of the novel bispecific antibodies described herein.

The effect of an anti-CD44-CTLA4 bispecific antibody on the level of CD71 on CD4⁺ and CD8⁺ T cells was assessed in 4 PBMC donors and compared to equimolar quantities of ipilimumab and nivolumab.

A grid of fusion proteins Fab-X and Fab-Y were created by diluting equimolar (1 μM) quantities of Fab-X (Fab-scFv) and Fab-Y (Fab-peptide) with specificity for CD44 and CTLA4 in TexMACS™ media (Miltenyi Biotec®) containing 100 U/mL penicillin/100 μg/mL streptomycin and 5% human AB Serum (Sigma-Aldrich). The Fab-X and Fab-Y fusion proteins were incubated together for 1 hour (in a 37° C., 5% CO₂ environment), at a final concentration of 500 nM. Negative control wells containing TexMACS™ media only were also generated alongside the Fab-X and Fab-Y wells. Ipilimumab and Nivolumab were diluted to 500 nM in TexMACS™ media containing 100 U/mL penicillin/100 μg/mL streptomycin and 5% human AB Serum (Sigma-Aldrich).

During this time, cryopreserved human PBMC isolated from platelet leukapheresis cones were thawed and washed in TexMACS™ media (containing 100 U/mL penicillin/100 μg/mL streptomycin and 5% human AB Serum (Sigma-Aldrich)) and resuspended in 10 mL PBS (ThermoFisher). 10 μL of 5 mM CellTrace™ Violet solution was added to the sample and mixed well by inversion. The cells were incubated at 37° C. for 20 minutes and washed by the addition of 45 mL PBS containing 10% heat inactivated foetal ovine serum (ThermoFisher). The cells were incubated for a further 5 minutes before centrifugation at 400×g for 5 minutes. The waste was removed, and the cells resuspended at 2.5×10⁶ cells/mL. The PBMC were then seeded into 96-well U-bottom tissue culture plates (Costar®) at 60 μL/well (1.5×10⁵ PBMC). A total of 20 μL of Fab-X/Fab-Y bispecific antibodies were transferred to the plates containing 60 μL PBMC. The PBMC were then either left unstimulated by the addition of 20 μL of media or stimulated with 20 μL of soluble antigen staphylococcal enterotoxin B; SEB (100 ng/mL final concentration). This resulted in a final assay concentration of Fab-X/Fab-Y bispecific antibodies of 100 nM. The plates were then returned to a 37° C., 5% CO₂ environment for 6 days.

After 6 days the plates were centrifuged at 500×g for 5 minutes at 4° C. Cell culture conditioned media was transferred from the cell pellets to fresh plates and frozen at ⁻80° C. Cells were washed with 60 μL FACS buffer by centrifugation, followed by resuspension of the pellets by shaking the plates at 1800 rpm for 30 seconds. The cells were stained with a cocktail of fluorescently labelled antibodies as listed in Table 2 and incubated at 4° C. in the dark for 30 minutes. After this time, cells were washed with FACS buffer, and fixed with 2% paraformaldehyde for one hour at 4° C. before the addition of 150 μL FACS buffer. The plates were centrifuged at 500×g for 5 minutes, the fixation buffer aspirated to waste and the cells resuspended in a residual volume of 15 μL for acquisition on the iQue® Screener Plus (IntelliCyt®).

The data analysis software package ForeCyt™ (IntelliCyt®) was used to exclude CD14⁺ monocytes, followed by the identification of the CD4⁺ and CD8⁺ T cells. For each cell population the cellular expression of CD71 was measured as reported median fluorescent intensity values. The data were then used to calculate the log 2 fold changes of expression relative to control well values.

TABLE 2

| Epitope | Fluorophore | Clone | Source | Dilution |
|---|---|---|---|---|
| CD14 | PE/Cy7 | M5E2 | BD Pharmagen ® | 80 |
| CD8 | BV605 | RPA-T8 | BioLegend ® | 40 |
| CD4 | FITC | RPA-T4 | BioLegend ® | 80 |
| CD71 | PE | M-A712 | BioLegend ® | 160 |
| Live/Dead | Near IR | | Thermo Scientific ® | 1000 |

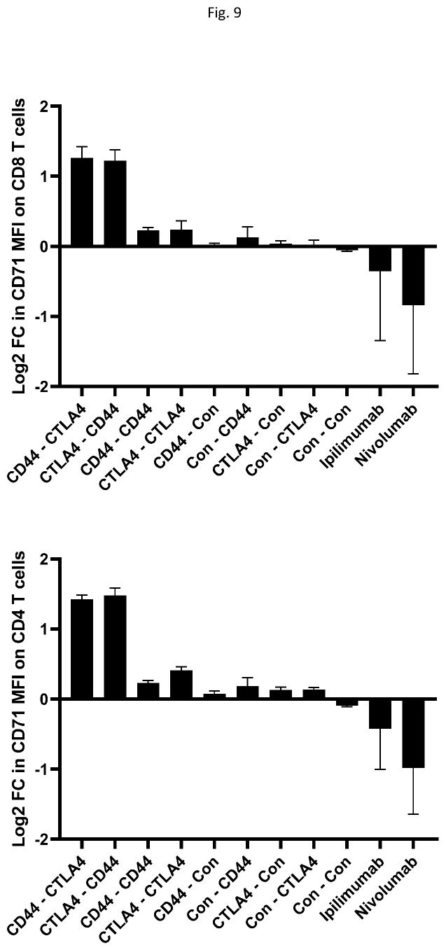

In combination with SEB stimulation over a 6-day period, CD44-CTLA4 bispecific antibodies led to an increase in the level of the activation marker CD71 on both CD8⁺ and CD4⁺ T cells (FIG. 9). Such increase was not observed for either the bivalent or monovalent controls antibodies. Neither Ipilimumab nor Nivolumab resulted in an increase in CD71 expression on the surface of T cells, with 1 donor displaying reduced CD71 levels.

### Example 4: Identification of Further CTLA4 Variable Regions as a Bispecific Antibody Complex with Anti-CD44

To investigate the reproducibility of activity of the CD44-CTLA4 bispecific antibody with different CTLA4 variable region sequences, 22 different anti-CTLA4 antibodies where tested in combination with anti-CD44 in an IL-2 secretion assay under SEB stimulated conditions.

A grid of fusion proteins Fab-X and Fab-Y were created by diluting equimolar (1 μM) quantities of Fab-X (Fab-scFv) with specificity for CD44 and Fab-Y (Fab-peptide) with 22 variable regions with specificity for CTLA4 plus negative control). Fusion proteins were prepared in TexMACS™ media (Miltenyi Biotec®) containing 100 U/mL penicillin/100 μg/mL streptomycin. The Fab-X and Fab-Y fusion proteins were incubated together for 1 hour (in a 37° C., 5% CO₂ environment), at a final concentration of 500 nM. Negative control wells containing TexMACS™ media only were also generated alongside the Fab-X and Fab-Y wells.

During this time, cryopreserved human PBMC isolated from platelet leukapheresis cones were thawed and washed in TexMACS™ media and resuspended at 2.5×10⁶ cells/mL. The PBMC were then seeded into 96-well U-bottom tissue culture plates (Costar) at 60 μL/well (1.5×10⁵ PBMC).

A total of 20 μL of Fab-X and Fab-Y complexes were transferred to the plates containing 60 μL PBMC in triplicate. The PBMCs were then either left unstimulated by the addition of 20 μL of TexMACS™ media or stimulated with 20 μL of SEB (1 μg/mL final concentration). This resulted in a final assay concentration of Fab-X and Fab-Y complexes of 100 nM. The plates were then returned to a 37° C., 5% CO₂ environment for 48 hours.

After 48 hours the plates were centrifuged at 500×g for 5 minutes at 4° C. Cell culture conditioned media was transferred from the cell pellets to fresh plates and frozen at −80° C. On the day of analysis, the conditioned media were thawed and diluted 20-fold in RPMI. The diluted conditioned media were then assayed for levels of IL-2 by bead ELISA. A standard curve for recombinant IL-2 was also generated allowing for the quantification of the IL-2 within the conditioned media.

The data analysis software package ForeCyt™ (IntelliCyt®) was used to measure the median fluorescent intensity values for the IL-2 detection beads. The log 2 fold changes of IL-2 concentration were calculated relative to control well values.

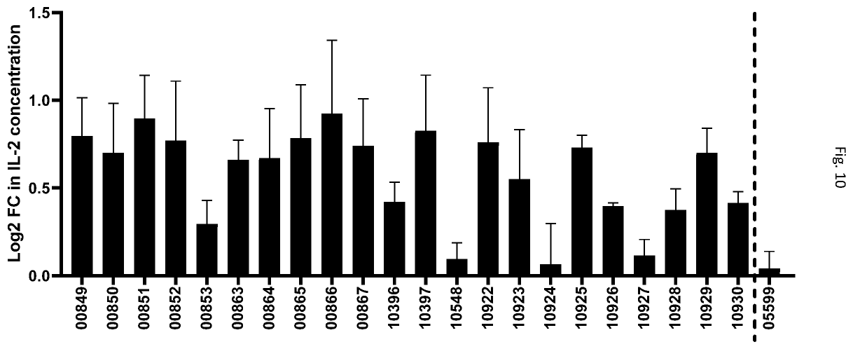

SEB induced the secretion of IL-2 into the conditioned media of the culture as shown previously, and the CD44 Fab-X, control (5599) Fab-Y fusion protein had no effect on the level of IL-2 detected. However, 14 of the 22 CTLA-4 variable regions tested gave increases in IL-2 of over 0.5 log 2 fold change from the SEB stimulus alone. A further 5 variable regions produced detectable increases below 0.5 log 2 fold change, while the remaining three variable regions caused no increase in IL-2 secretion (FIG. 10).

### Example 5: Evaluation of the of the Ability of CD44-CTLA4 Bispecific Antibody to Activate T Cells in the Presence of Excess T Regulatory Cells

To demonstrate the effect of anti-CD44-CTLA4 bispecific antibodies on activation of T cells in the presence of highly suppressive T regulatory cells (T_{REG}), 4 donors were assayed whereby isolated T effector (T_{EFF}) cells were cocultured with T_{REG} at a ratio of 1 T_{REG}:4 T_{EFF} under T cell stimulating conditions.

In vitro expanded T_{REG} were used in this assay. To obtain expanded T_{REG}, human PBMC were isolated from platelet leukapheresis cones by Ficoll® density gradient centrifugation according to standard procedures. T_{EFF} and T_{REG} were isolated by MACS® magnetic cell separation using the CD4⁺ CD25⁺ Regulatory T Cell Isolation Kit, (Miltenyi Biotec®), following the manufacturer's protocol. To test the purity of isolated T_{EFF} and T_{REG}, cells were stained for surface markers CD4, CD25, CD127 and for the transcription factor FOXP3. After isolation, T_{EFF} cells were frozen in FBS with 10% DMSO. T_{REG} were seeded into 96-well U-bottom tissue culture plates (Corning Inc.) in expansion medium comprised of X-VIVO™ 15 medium (Lonza) containing 10% human AB serum (Sigma Aldrich®), 1 mM N-acetylcysteine (Sigma Aldrich®), 100 nM Rapamycin (Sigma Aldrich®) and 300 U/mL recombinant human IL-2 (PeproTech®) at 100 μL/well (1.0×10⁵ T_{REG}). 100 μL/well of Dynabeads™ (Human T-Activator CD3/CD28 for T Cell Expansion and Activation) were added to each well containing T_{REG} to reach a final ratio of 1 T_{REG}:4 beads. Plates were kept in a 37° C., 5% CO₂ environment. On day 3, the medium was carefully removed and replaced with freshly made expansion medium. On day 6, medium was carefully removed, cells were resuspended, pooled, counted and reseeded into 96-well U-bottom tissue culture plates (Corning Inc.) at 200 mL/well (1.0×10⁵ T_{REG}). The plates were then returned to a 37° C., 5% CO₂ environment until day 10 when they were collected into a 50 mL Falcon tube, centrifuged at 400×g for 5 minutes and resuspended in resting medium, consisting of X-VIVO™ 15 medium containing 5% human AB serum and 100 U/mL recombinant human IL-2. The tube was then placed onto a magnetic separator to remove the Dynabeads™. The bead-free cell suspension was transferred to a new tube, cells were counted, diluted in resting medium to 1.0×10⁶ cells/mL, seeded at 0.5 mL/cm² and returned to a 37° C., 5% CO₂ environment. On day 14, expanded T_{REG} were collected, centrifuged at 400×g for 5 minutes and frozen in FBS (Life Technologies™) with 10% DMSO (Sigma Aldrich®). Two days prior to setting up the suppression assay, expanded T_{REG} were thawed, washed in X-VIVO™ 15 medium and resuspended in resting medium at 1.0×10⁶ cells/mL then seeded at 0.5 mL/cm². Cells were rested for 48 hours at 37° C., 5% CO₂. On the following day, T_{EFF} cells were thawed, washed in X-VIVO™ 15, resuspended in resting medium at 5.0×10⁶ cells/mL before plating at 0.5 mL/cm² and culturing at 37° C., 5% CO₂.

On the day of the assay, a grid of fusion proteins Fab-X and Fab-Y were created by diluting equimolar (1 μM) quantities of Fab-X (Fab-scFv) and Fab-Y (Fab-peptide) with specificity for CTLA-4 and CD44 in TexMACS™ medium (Miltenyi Biotec®) containing 5% AB human serum (Sigma-Aldrich®) and 100 U/mL penicillin/100 μg/mL streptomycin (suppression media). Ipilimumab and Nivolumab were generated in the same manner at a final concentration of 500 nM. The Fab-X and Fab-Y fusion proteins were incubated together for 1 hour in a 37° C., 5% CO₂ environment, at a final concentration of 500 nM. Negative control wells containing suppression media only were generated alongside the Fab-X and Fab-Y wells.

During this time, T_{EFF} were harvested, centrifuged at 400×g for 5 minutes, resuspended in 10 mL PBS and labelled with CellTrace™ Violet (CTV) (Thermo Scientific®) for 20 minutes at 37° C., 5% CO₂ at a dilution of 1:1000. The reaction was stopped by adding 40 mL of PBS with 10% FBS to the cells and incubating at 37° C., 5% CO₂ for 5 minutes. T_{EFF} were centrifuged, resuspended in suppression medium, counted and diluted at 5.0×10⁵ cells/mL. T_{REG} were collected, centrifuged at 400×g for 5 minutes, resuspended in suppression medium, counted and diluted at 5.0×10⁵ cells/mL. T_{EFF} and T_{REG} were cocultured at 1 T_{REG}:4 T_{EFF} ratio, in the presence of T_{REG} Suppression Inspector beads (Miltenyi Biotec®) at a 1 Bead:1 cell ratio, in a volume of 80 μL/well. A total of 20 μL of Fab-X/Fab-Y bispecific antibodies or ipilimumab and nivolumab were transferred to the plates containing 80 μL T_{REG}:T_{EFF}. This resulted in a final assay concentration of antibodies of 100 nM. The plates were incubated for 5 days in a 37° C., 5% CO₂ environment.

After 5 days, the plates were centrifuged at 500×g for 5 minutes. Cell culture conditioned media was transferred from the cell pellets to fresh plates and frozen at ⁻80° C. Cells were washed with 200 μL FACS buffer once by centrifugation at 500×g for 5 minutes, followed by resuspension of the pellets by shaking the plates at 1800 rpm for 15 seconds. The cells were stained with 20 μL of fluorescently labelled antibody cocktail as listed in Table 3 and incubated at room temperature in the dark for 30 minutes. After this time, cells were washed once with FACS buffer, before fixing with 2% paraformaldehyde diluted in PBS for 1 hour at 4° C. in the dark. Cells were then washed once with 150 μL FACS buffer by centrifugation at 500×g for 5 minutes, buffer was aspirated, and the cells resuspended in a residual volume of 15 μL for acquisition on the iQue® Screener Plus (IntelliCyt®).

The data analysis software package ForeCyt™ (IntelliCyt®) was used to gate on CD4⁺ CTV⁺ T_{EFF} cells and for this population the cellular expression of CD25 and CD71 were measured as reported median fluorescent intensity (MFI) values. The data were then used to calculate the log 2 fold changes of expression relative to assay ratio control well values.

TABLE 3

| Epitope | Fluorophore | Clone | Source | Dilution |
|---|---|---|---|---|
| CD4 | PE | HCD56 | BioLegend ® | 10 |
| CD25 | APC | M5E2 | BD Pharmagen ® | 10 |
| CD71 | FITC | M-A712 | BD Pharmagen ® | 10 |
| Live/Dead | Near IR | | Thermo Scientific ® | 1000 |

Co-culture of the expanded T_{REG} cells with the T_{EFF} reduced the level of CD25 and CD71 expressed in the surface of the T_{EFF} cells, suggesting a suppressive environment. The CD44-CTLA-4 bispecific antibody led to an increase in the level of CD25, and to a lesser extent CD71, on the surface of the T_{EFF} in this suppressive co-culture environment. The CD44 bivalent and monovalent controls also resulted in a detectable, but smaller increase in activation marker expression, suggesting some effect by an anti-CTL4 antibody alone. However, no increase was identified with the CTLA-4 bivalent and monovalent controls or Ipilimumab. Nivolumab also did not show any increase.

These results suggest that the anti-CTL4 and CD44 bispecific antibody according to the present invention may be able to stimulate T cell activation in the present of regulatory T cells, eventually leading to reverse of immune suppression in the cancer microenvironment.

1. An antibody which comprises a first antigen-binding portion binding CTLA4 and a second antigen-binding portion binding CD44.

2. The antibody according to claim 1, wherein each of the antigen-binding portions is a monoclonal antigen-binding portion.

3. The antibody according to claim 1 or claim 2, wherein each of the antigen-binding portions is independently selected from a Fab, a Fab′, a scFv or a VHH.

4. The antibody according to claim 1 or claim 2, wherein the antigen-binding portions are the antigen-binding portions of an IgG.

5. The antibody according to any one of the preceding claims wherein the antibody is chimeric, human or humanised, preferably the antibody is humanised.

6. The antibody according to any one of the preceding claims wherein the antibody comprises a heavy chain constant region selected from an IgG1, an IgG2, IgG3 or an IgG4 isotype, or a variant thereof.

7. The antibody according to anyone of the preceding claims, wherein the antibody further comprises at least an additional antigen-binding portion.

8. The antibody according to claim 7, wherein the additional antigen-binding portion is capable of increasing the half-life of the antibody.

9. The antibody according to claim 8, wherein the additional antigen-binding portion binds albumin, preferably human serum albumin.

10. The antibody according to any one of the preceding claims wherein the first antigen-binding portion binding CTLA4 comprises a first heavy chain variable region and a first light chain variable region and the second antigen-binding portion binding CD44 comprises a second heavy chain variable region and a second light chain variable region and wherein:
a. The first heavy chain variable region comprises a CDR-H1 comprising SEQ ID NO: 21, a CDR-H2 comprising SEQ ID NO: 22 and a CDR-H3 comprising SEQ ID NO: 23; and
b. The first light chain variable region comprises a CDR-L1 comprising SEQ ID NO: 24, a CDR-L2 comprising SEQ ID NO: 25 and a CDR-L3 comprising SEQ ID NO: 26; and
c. The second heavy chain variable region comprises a CDR-H1 comprising SEQ ID NO: 27, a CDR-H2 comprising SEQ ID NO: 28 and a CDR-H3 comprising SEQ ID NO: 29; and
d. The second light chain variable region comprises a CDR-L1 comprising SEQ ID NO: 30, a CDR-L2 comprising SEQ ID NO: 31 and a CDR-L3 comprising SEQ ID NO: 32;
or
e. The first heavy chain variable region comprises SEQ ID NO: 33 and the first light chain variable region comprises SEQ ID NO: 35; and the second heavy chain variable region comprises SEQ ID NO: 37 and second light chain variable region comprises SEQ ID NO: 39;
or
f. The first heavy chain variable region is encoded by a nucleotide sequence comprising SEQ ID NO: 34 and the first light chain variable region is encoded by a nucleotide sequence comprising SEQ ID NO: 36; and the second heavy chain variable region is encoded by a nucleotide sequence comprising SEQ ID NO: 38 and second light chain variable region is encoded by a nucleotide sequence comprising SEQ ID NO: 40.

11. A pharmaceutical composition comprising the antibody according to any one of the preceding claims and one or more pharmaceutically acceptable excipients.

12. The antibody according to any one of claims 1 to 10 or the pharmaceutical composition according to claim 11 for use in therapy.

13. The antibody according to any one of claims 1 to 10 or the pharmaceutical composition according to claim 11 for use in the treatment of cancer and/or an infectious disease.

14. The antibody for use according to claim 13, wherein the antibody or the composition are for use in the treatment of cancer concomitantly or sequentially to one or more additional cancer therapies.

15. The antibody for use according to any one of claims 12 to 14, wherein the antibody stimulates T cell activation in the presence of regulatory T cells; wherein activation preferably reverses, at least in part or in full, immune suppression.

16. A method for treating a subject afflicted with cancer and/or an infectious disease, comprising administering to the subject a pharmaceutically effective amount of an antibody according to any one of claims 1 to 10 or a pharmaceutical composition according to claim 11.

17. The method according to claim 16, wherein the antibody or the composition are administered concomitantly or sequentially to one or more additional cancer therapies.

18. The method according to any one of claim 16 or 17, wherein the antibody stimulates T cell activation in the presence of regulatory T cells; wherein activation preferably reverse, at least in part or in full, immune suppression.

19. Use of an antibody according to any one of claims 1 to 10 or a pharmaceutical composition according to claim 11 in the manufacture of a medicament for treating cancer.

20. The use according to claim 19, wherein the antibody or the composition are administered concomitantly or sequentially to one or more additional cancer therapies.

21. The use according to any one of claim 19 or 20, wherein the antibody stimulates T cell activation in the presence of regulatory T cells; wherein activation preferably reverse, at least in part or in full, immune suppression.


SEQUENCE LISTING (40 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 741 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSATGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 3 - protein, 29 aa, Homo sapiens
MDKFWWHAAWGLCLVPLSLAQIGVGRRKS

SEQ ID NO: 4 - protein, 711 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGALMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 5 - protein, 699 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATSTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 6 - protein, 734 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 7 - protein, 699 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTTASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 8 - protein, 713 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTRNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 9 - protein, 674 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 10 - protein, 675 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRS

SEQ ID NO: 11 - protein, 493 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 12 - protein, 429 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 13 - protein, 361 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 14 - protein, 425 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 15 - protein, 396 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTRRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 16 - protein, 294 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRS

SEQ ID NO: 17 - protein, 668 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGALMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTTASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 18 - protein, 691 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTTASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 19 - protein, 340 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 20 - protein, 139 aa, Homo sapiens
MDKFWWHAAWGLCLVPLSLAQIDLNITCRFAGVFHVEKNGRYSISRTEAADLCKAFNSTLPTMAQMEKALSIGFETCSLHCSQQSKKVWAEEKASDQQWQWSCGGQKAKWTQRRGQQVSGNGAFGEQGVVRNSRPVYDS

SEQ ID NO: 21 - protein, 11 aa, Artificial Sequence
  CDR-H1 VR0851
GIDFSDYYYMC

SEQ ID NO: 22 - protein, 18 aa, Artificial Sequence
  CDR-H2 VR0851
CIYGASSDSTYYASWAKG

SEQ ID NO: 23 - protein, 11 aa, Artificial Sequence
  CDR-H3 VR0851
SDYLYDYAMNL

SEQ ID NO: 24 - protein, 11 aa, Artificial Sequence
  CDR-L1 VR0851
QASQTINTYLS

SEQ ID NO: 25 - protein, 7 aa, Artificial Sequence
  CDR-L2 VR0851
SASTLAS

SEQ ID NO: 26 - protein, 12 aa, Artificial Sequence
  CDR-L3 VR0851
QSHYYIVSNGWP

SEQ ID NO: 27 - protein, 10 aa, Artificial Sequence
  CDR-H1 VR5479
GFSLSSYAMS

SEQ ID NO: 28 - protein, 16 aa, Artificial Sequence
  CDR-H2 VR5479
GIVSTGITYYASWAKG

SEQ ID NO: 29 - protein, 13 aa, Artificial Sequence
  CDR-H3 VR5479
ILYSGGSGNYLHL

SEQ ID NO: 30 - protein, 13 aa, Artificial Sequence
  CDR-L1 VR5479
QSSESVLSNNRFS

SEQ ID NO: 31 - protein, 7 aa, Artificial Sequence
  CDR-L2 VR5479
GASSLAS

SEQ ID NO: 32 - protein, 12 aa, Artificial Sequence
  CDR-L3 VR5479
AGYKSPTYDDWA

SEQ ID NO: 33 - protein, 120 aa, Artificial Sequence
  VH VR0851
QSLEESGGDLVKPGASLTLTCTASGIDFSDYYYMCWVRQAPGKGLELIACIYGASSDSTYYASWAKGRFTISKTSSTTVTLQMTSLTAADTATYFCVRSDYLYDYAMNLWGQGTLVTVSS

SEQ ID NO: 34 - DNA, 433 bp, Artificial Sequence
  VH nucl VR0851
aagcttcgaagccaccatggagactgggctgcgctggcttctcctggtcgctgtgctcaaaggtgtccagtgtcagtcgttggaggagtccgggggagatctggtcaagcctggggcatccctgacactcacctgcacagcctctggaatcgacttcagtgactactactacatgtgctgggtccgccaggctccagggaaggggctggagttgatcgcctgcatttatggtgctagtagtgatagcacatactacgcgagctgggcgaaaggccgattcaccatctccaaaacctcgtcgaccacggtgactctgcaaatgaccagtctgacagccgcggacacggccacctatttctgtgtgagaagtgactatctatatgattatgccatgaacttgtggggccaaggcaccctcgtcaccgtctcgagt

SEQ ID NO: 35 - protein, 110 aa, Artificial Sequence
  VL VR0851
DVVMTQTPASVEAAVGDIVTIKCQASQTINTYLSWYQQKPGQPPKLLIYSASTLASGVPSRFKGSGSGTDYTLTISDLECADAATYYCQSHYYIVSNGWPFGGGTEVVVE

SEQ ID NO: 36 - DNA, 418 bp, Artificial Sequence
  VL nucl VR0851
aagcttcgaagccaccatggacacgagggcccccactcagctgctggggctcctgctgctctggctcccaggtgccagatgtgatgttgtgatgacccagactccagcctccgtggaggcagctgtgggagacatagtcaccatcaagtgccaggccagtcagaccattaatacttacttatcctggtatcagcagaaaccagggcagcctcccaagctcctgatctattctgcatccactctggcatctggggtcccatcgcggtttaaaggcagtggatctgggacagattacactctcaccatcagcgacctggagtgtgccgatgctgccacttactactgtcaaagtcattattatattgttagtaatggttggcctttcggcggagggaccgaggtggtcgtcgaacgtacg

SEQ ID NO: 37 - protein, 118 aa, Artificial Sequence
  VH VR5479
QSLEESGGRLVRPDETLTLTCTVSGFSLSSYAMSWVRQAPGKGLEYIGGIVSTGITYYASWAKGRFTISKTSTTVTLKMTSLTTEDTATYFCVRILYSGGSGNYLHLWGQGTLVTVSS

SEQ ID NO: 38 - DNA, 427 bp, Artificial Sequence
  VH nucl VR5479
aagcttcgaagccaccatggagactgggctgcgctggcttctcctggtcgctgtgctcaaaggtgtccagtgtcagtcgctggaggagtccgggggtcgcctggtcaggcctgacgaaaccctgacactcacctgcacagtctctggattctccctcagtagctatgcaatgagctgggtccgccaggctccagggaaggggctggaatacatcggaggcattgttagtactggtatcacatactacgcgagctgggcgaaaggccgattcaccatctccaaaacctcgaccacggtgactctgaaaatgaccagtctgacaaccgaggacacggccacctatttctgtgtcagaatattatatagtggtggtagtggtaattaccttcacttgtggggccaaggcaccctggtcaccgtctcgagt

SEQ ID NO: 39 - protein, 112 aa, Artificial Sequence
  VL VR5479
ALVMTQTPSPVSAAVGGTVTISCQSSESVLSNNRFSWYQQKLGQPPKLLIYGASSLASGVPSRFKGSGSGTEFTLTISGVECDDAATYYCAGYKSPTYDDWAFGGGTEVVVK

SEQ ID NO: 40 - DNA, 424 bp, Artificial Sequence
  VL nucl VR5479
aagcttcgaagccaccatggacacgagggcccccactcagctgctggggctcctgctgctctggctcccaggtgccagatgtgcgcttgtgatgacccagactccatcccctgtgtctgcagctgtgggaggcacagtcaccatcagttgccagtccagtgagagtgttcttagtaataaccgcttttcctggtatcagcagaaactagggcaacctcccaagctcctgatctatggtgcatccagtctggcgtctggggtcccatcgcggttcaaaggcagtggatctgggacagagttcactctcaccatcagtggcgtggagtgtgacgatgctgccacttactactgtgcaggctataaaagtcctacctatgatgactgggctttcggcggagggaccgaggtggtggtcaaacgtacg